(19) United States
(12) Patent Application Publication
(10) Pub. No.: US 2021/0052731 A1
(43) Pub. Date: Feb. 25, 2021

(54) INORGANIC NANOPHOTOSENSITIZERS AND METHODS OF MAKING AND USING SAME

(71) Applicant: Cornell University, Ithaca, NY (US)

(72) Inventors: Ferdinand F.E. KOHLE, Tubingen (DE); Ulrich B. WIESNER, Ithaca, NY (US)

(21) Appl. No.: 17/052,103

(22) PCT Filed: May 2, 2019
(86) PCT No.: PCT/US2019/030495

§ 371 Date: Oct. 30, 2020

Related U.S. Application Data
(60) Provisional application No. 62/666,086, filed on May 2, 2018.

(51) Int. Cl. A61K 41/00; C01B 33/12; A61K 9/51

(52) U.S. Cl. CPC A61K 41/0057; C01B 33/12; B82Y 5/00; A61K 9/5192; A61K 9/5115

(57) ABSTRACT

Provided are nanoparticles surface functionalized with PEG groups and having one or more photosensitizer group. The PEG groups may be functionalized. The nanoparticles may also include therapeutic groups and/or targeting groups. The nanoparticles may be made by hydrolysis of a silica precursor and, optionally, an alumina precursor, in various aqueous reaction mediums. The nanoparticles may be used in photodynamic therapy methods. The methods may also include imaging of an individual.

### CROSS REFERENCE TO RELATED APPLICATIONS

This application claims priority to U.S. Provisional Application No. 62/666,086, filed on May 2, 2018, the disclosure of which is hereby incorporated by reference.

### STATEMENT REGARDING FEDERALLY SPONSORED RESEARCH OR DEVELOPMENT

This invention was made with government support under Grant No. DE-SC0010560 awarded by the Department of Energy. The government has certain rights in the invention.

### FIELD OF THE DISCLOSURE

The disclosure generally relates to ultrasmall nanoparticles with photosensitizers and methods of making and using same. More particularly, the disclosure generally relates to ultrasmall silica and aluminosilicate nanoparticles with photosensitizers used for photodynamic therapy.

### BACKGROUND OF THE DISCLOSURE

Photodynamic therapy (PDT) presents an alternative non-invasive therapeutic modality for the treatment of cancer and other diseases. PDT relies on cytotoxic singlet oxygen that is locally generated through energy transfer between a photosensitizer and molecularly dissolved triplet oxygen. To minimize side-effects, i.e. damage of healthy tissue, targeted delivery to places of disease, high local photosensitizer concentrations, high singlet oxygen quantum yield, and rapid post-treatment clearance of photosensitizers are desired. 10005) Silica nanoparticles (SNPs) have attracted interest for potential therapeutic/diagnostic applications due to their large surface-area, inertness and high bio-compatibility. However, most SNPs are >10 nm in size.

Particles >12 nm are not effectively cleared from the body in vivo and unfavorably distribute to the liver and other organs/tissues, potentially exposing these tissues to toxic elements (especially if these >10 nm SNPs are modified with drugs and/or radioactivity). Particles about 8 nm in diameter reside in the body for about a day, 10-11 nm about 3-5 days, but if greater than 12 nm do not clear or clear very slowly.

Currently, ultrasmall inorganic nanoparticles are of rapidly increasing interest as nanomedicines for cancer theranostics. Some organic based nanomedicines are already more competitive than conventional chemotherapy drugs due to multifunctionality and multivalency effects. Inorganic nanoparticles further diversify the building elements of nanomedicines and may provide advantages associated with their intrinsic physical properties and lower manufacturing costs. Safe translation of nanoparticles from the laboratory to the clinic requires overcoming a number of substantial scientific and regulatory hurdles. The most important criteria are favorable biodistribution and its time evolution (pharmacokinetics, PK) profiles. The size threshold for renal clearance is below 10 nm. Until today only a small number of inorganic nanoparticle platforms have been synthesized with sizes below 10 nm allowing for efficient renal clearance. Among those only <10 nm sized polyethylene glycol coated (PEGylated) fluorescent core-shell silica nanoparticles (SNPs) referred to as Cornell dots or simply C dots have been approved by the U.S. Food and Drug Administration (FDA) as an investigational new drug (IND) for first in-human clinical trials. Although the first clinical trial results with melanoma patients are encouraging, several synthesis challenges remain for such sub 10 nm sized fluorescent organic-inorganic hybrid SNPs.

First, all previous C dot-type SNP synthesis efforts followed a modified Stöber process in which alcohol was used as solvent. For materials for use in biological or clinical applications, however, water as a reaction medium would be preferred. It would greatly simplify synthesis and cleaning protocols leading to less volatile waste, thereby rendering particle production substantially faster and more cost effective. Furthermore, although the Stöber process is widely used to produce SNPs with diameters from tens of nm to microns, particle sizes of 10 nm and below are at the limit of size control of this synthesis process due to reaction kinetics limitations in alcohol.

Second, covalently covering silica particle surfaces with PEG can be tricky as the loss of surface charge during PEGylation may result in particle aggregation or at least broadening of the particle size distribution. This effect is more pronounced for ultrasmall particles due to the increase of particle surface energy, and thus limits the particle monodispersity and size control ability.

Third, as a result of the negative surface charge of silica above its isoelectric point at pH 2-3, covalent encapsulation efficiencies for silane-conjugated organic fluorescent dyes with negatively charged groups into SNPs are low as a result of electrostatic repulsion between silica and fluorophore. This is particularly true for near-infrared (NIR) emitting dyes most desirable for imaging applications in living tissue. NIR dyes have large delocalized π-electron systems and to be soluble in water typically require multiple negatively charged functional groups (e.g., sulfates) on their periphery. Low incorporation efficiencies are a problem for these dyes as their typical costs are of order $200-$300 per mg and re-use of typically employed silane-dye conjugates after the initial synthesis is problematic.

Finally, no inorganic elemental compositions other than silica have been reported for <10 nm sized fluorescent SNPs and core-shell SNPs. In particular, compositions are of interest leading to higher rigidity of the organic dye environments as increases in rigidity have directly been correlated with increases in per dye fluorescence yield as a result of decreases in non-radiative rates.

All these challenges suggest revisiting the original fluorescent core-shell SNP (C dot) synthesis in order to systematically develop a water based approach to <10 nm organic-inorganic hybrid dots with improved size control, previously unknown compositions, and enhanced performance characteristics.

Different NP-based systems have been described in the literature, e.g., PEGylated liposomes, polymeric NPs, iron oxide NPs, and gold NPs. However, these systems suffer from various limitations.

Photodynamic therapy (PDT) emerged as a minimally invasive and minimally toxic therapeutic modality for the treatment of cancer and other diseases. The principle of PDT can generally be described in four steps: A photosensitizer (PS) is localized around diseased tissue (step 1), and activated by a light source (step 2). The absorbed photon energy leads to the generation of highly reactive singlet oxygen, ¹O₂ (step 3), causing oxidative stress and cellular damage, eventually initiating cell death mechanisms such as necrosis and/or apoptosis in the local environment of the PS (step 4). These steps impose chemical, photophysical, and structural requirements onto PDT probes.

Different NP-based systems, organic, inorganic, and organic-inorganic hybrid, have been described in the literature, including PEGylated liposomes, polymeric NPs, iron oxide NPs, or gold NPs. While numerous NPs systems are able to load large amounts of PS molecules, few NP platforms combine the necessary ease of chemical functionalization with precise particle size control on the sub-10 nm length scale, to meet the requirements for successful clinical translation and synthesis scale-up. While metal-organic framework NPs in principle offer these capabilities, targeted delivery and systematic in vitro and in vivo studies on NP activity and fate demonstrating favorable characteristics are still lacking.

Based on the foregoing there exists an ongoing and unmet need for improved nanoprobes for photodynamic therapy.

### SUMMARY OF THE DISCLOSURE

The present disclosure provides inorganic nanoparticles. The nanoparticles are also referred to herein as ultrasmall nanoparticles. The present disclosure also provides methods of making the nanoparticles and uses of the nanoparticles.

In an aspect, the present disclosure provides inorganic nanoparticles. The nanoparticles may be silica nanoparticles and aluminosilicate nanoparticles. The nanoparticles comprise one or more photosensitizer. The photosensitizer(s) are covalently bound to the silica matrix or aluminosilicate matrix of the nanoparticle and/or covalently bound to an exterior surface of the nanoparticle and/or are part of a PEG group. The nanoparticles comprise a silica core or an aluminosilicate core and the silica core or an aluminosilicate core comprises one or more PEG groups and/or functionalized PEG groups covalently bound to a surface of the silica core or an aluminosilicate core. A nanoparticle may be present in a composition comprising a plurality of nanoparticles of the present disclosure.

A nanoparticle can comprise various photosensitizers (PSs) (e.g., photosensitizer groups). Non-limiting examples of photosensitizers include psoralens (e.g., 5-methoxypsoralen and the like), porphyrinoids (e.g., porphyrins, chlorins, bacteriochlorins, phthalocyanines, and naphthalocyanines, and the like), phenothiazines (e.g., methylene blue (e.g., MB2), toluidine blue, and the like), cyanines (e.g., merocyanine 540 and the like), curcuminoids (e.g., curcumin and the like), BODIPY (e.g., BODIPY 650/665 and the like), xanthenes (e.g., Rose Bengal and the like), 4,5-dibromorhodamine methyl ester (TH9402), derivatives or analogs thereof, groups derived therefrom, and combinations thereof.

A nanoparticle may be functionalized with various groups. The groups may be covalently bound to a surface of the nanoparticle and/or part of a PEG group covalently bound to a surface of the nanoparticle. For example, a nanoparticle is functionalized with groups chosen from peptides (natural or synthetic), cyclic peptides (e.g., cyclic-RGD and derivatives thereof, alpha-MSH and derivatives thereof, and the like), nucleic acids (e.g., single stranded or double stranded DNA, various forms of RNA (e.g., siRNA, and the like), lipids, carboyhydrates (e.g., oligosaccharides, polysaccharides, sugars, and the like), groups comprising a radio label (e.g., ¹²⁴I, ¹³¹I, ²²⁵Ac or ¹⁷⁷Lu, ⁸⁹Zr, ⁶⁴Cu, and the like), antibodies, antibody fragments, groups comprising a reactive group (e.g., a reactive group that can be further conjugated, for example, via click chemistry, to a molecule such as, for example, a pharmaceutical product (e.g., a drug molecule, which may be a toxic drug molecule, a small molecule inhibitor (e.g., gefitinib, and the like)), and combinations thereof.

In an aspect, the present disclosure provides a method of making nanoparticles. The methods are based on use of aqueous reaction medium (e.g., water). The nanoparticles can be surface functionalized with polyethylene glycol groups (e.g., PEGylated). The methods as described herein may be linearly scaled up, e.g., from 10 ml reaction to 1000 ml or greater without any substantial change in product quality. This scalability is important for large scale manufacture of the nanoparticles. The methods are carried out in an aqueous reaction medium (e.g., water). The nanoparticles can be PEG functionalized by various methods. Methods of PEG functionalization are known in the art. Combinations of PEG functionalization methods may be used.

In an aspect, the present disclosure provides compositions comprising nanoparticles of the present disclosure. The compositions can comprise one or more types (e.g., having different average size and/or one or more different compositional feature) of nanoparticles. A composition may comprise a nanoparticle and a pharmaceutically acceptable carrier. A composition may comprise a plurality of nanoparticles from a single reaction mixture or a plurality of nanoparticles from two or more different reaction mixtures. A composition may comprise nanoparticle having the same photosensitizer (incorporated in the same or different way) or a combination of two or more structurally distinct photosensitizers (each incorporated in the same or different way). The nanoparticles or nanoparticle cores of the nanoparticles in a composition can have a variety of sizes.

In various aspects, the present disclosure provides uses of nanoparticles and/or compositions of the present disclosure. Non-limiting examples of uses of the nanoparticles and/or compositions of the present disclosure include imaging methods and photodynamic therapy (PDT) methods, and the like.

Nanoparticle(s) or composition(s) of the present disclosure can be used in various PDT methods. A PDT method may further comprise visualization of the cancer after administration of the nanoparticle or the composition. In an example, the visualization is carried out using fluorescence imaging. A method of the present disclosure can be used to treat an individual with (e.g., diagnosed with) cancer.

In an aspect, the present disclosure provides kits. A kit comprises one of more nanoparticle and/or one or more composition of the present disclosure. The composition(s) may be pharmaceutical compositions. A kit may comprise one or more nanoparticle of the present disclosure and/or one or more composition of the present disclosure, and instructions for use of the nanoparticle(s) and/or composition(s) for treatment of (e.g., administration to) an individual.

### BRIEF DESCRIPTION OF THE FIGURES

For a fuller understanding of the nature and objects of the disclosure, reference should be made to the following detailed description taken in conjunction with the accompanying figures.

FIG. 1 depicts synthesis of sub-10 nm silica nanoparticles. (A) Simplified Jablonski Scheme illustrating the creation of reactive singlet oxygen, ¹O₂. ¹PS denotes the singlet ground state, ¹PS* the electronically excited singlet state, and ³PS* the electronically excited triplet state of a photosensitizer. ³O₂ denotes the triplet ground state of molecularly dissolved oxygen. (B) Precursor molecules for the synthesis of sub-10 nm silica nanoparticles, showing the methylene blue derivate MB2-silane, the rhodamine dye TMR-silane, tetramethyl orthosilicate (TMOS), and polyethylene glycol-silane (PEG-silane). (C) Schematic representation of two different designs of functionalized photosensitizing sub-10 nm silica nanoparticles (center). Design 1: Covalent encapsulation of one or more MB2 molecules in the silica matrix (PEG-MB2-C′ dots). Design 2: Particle surface functionalization with one or more MB2 molecules (MB2-PEG-C′ dot). (D) Targeting moiety cyclo(Arg-Gly-Asp-D-Tyr-Cys) (cRGDyC).

FIG. 2 depicts fluorescence correlation spectroscopy of nanoparticles. (A) and (C) FCS autocorrelation curve of MB2-PEG-TMR-C′ dot (design one) and absorption spectra before and after TMR surface functionalization as compared to free TMR dye and MB2 photosensitizer. (B) and (D) FCS autocorrelation curves of PEG-TMR-C′ dots and TMR-PEG-MB2-C′ dots (design two) and absorption spectra before and after MB2 surface functionalization as compared to free TMR dye and MB2 photosensitizer.

FIG. 3 depicts photosensitizing measurement of nanoparticles. (A) Schematic representation of a photosensitizing measurement using 1,3-diphenylisobenzofuran (DPBF) as a singlet oxygen, ¹O₂, sensor. Absorption of a solution containing TMR-PEG-MB2-C′ dots and DPBF irradiated at 635 nm for 60 s in intervals of 5 and 10 s (see legend and black time arrow). (B) Comparative ¹O₂ generation of methylene blue, TMR-PEG-MB2-C′ dots, and MB2-PEG-TMR-C′ dots.

FIG. 4 depicts absorption spectrum. (A) Intensity matched absorption spectra of PEG-MB2-C′ dots and c(RGDyC)-PEG-MB2-C′ dots. (B) Difference spectrum of the spectra in (A). (C) Photosensitizing measurement of intensity matched PEG-MB2-C′ dots and c(RGDyC)-PEG-MB2-C′ dots. (D) Intensity matched absorption spectra of MB2-PEG-C′ dots and MB2-c(RGDyC)-PEG-C′ dots. (E) Difference spectrum of the spectra in (D). (F) Photosensitizing measurement of intensity matched MB2-PEG-C′ dots and MB2-c(RGDyC)-PEG-C′ dots.

FIG. 5 depicts methylene blue absorption spectra. Methylene blue (MB) and MB2 absorption spectra. A minor bathochromic shift (red-shift) from 665 to 667 nm is noticeable. The inset shows the chemical structure of MB with auxochrome groups circled in blue.

FIG. 6 depicts fluorescence emission spectra of dyes and photosensitizers and nanoparticles comprising dyes and nanoparticles comprising photosensitizers. Fluorescence emission spectra of Cy5 dye, PEG-Cy5-C′ dots, MB2, PEG-MB2-C′ dots, and MB2-PEG-C′ dots. The concentration of Cy5 and MB2 was matched. The emission spectra were normalized for the emission of MB2. The inset shows the enlarged emission of MB2, PEG-MB2-C′ dots, and MB2-PEG-C′ dots, showing a more than one order of magnitude brightness difference between the Cy5 and PEG-Cy5-C′ dots and MB2, PEG-MB2-C′ dots, and MB2-PEG-C′ dots. Cy5 particles show the typical emission enhancement that is observed upon dye encapsulation in a silica matrix as compared to the free dye in solution. The emission of PEG-MB2-C′ dots as compared to MB2-PEG-C′ dots is slightly larger, however both particles demonstrate a reduction of emission as compared to free MB2. This can likely be associated with the increased shoulder of MB2 in the particles.

FIG. 7 depicts chemical structures. (A) Chemical structure of the targeting moiety precursor (cRGDyC)-PEG(12)-silane. (B) Chemical structure of (3-mercaptopropyl)trimethoxysilane (MPTMS).

FIG. 8 depicts GPC (gel permeation/size-exclusion chromatography) elugrams. (A) GPC elugrams of PEG-MB2-C′ dots (i) and TMR-PEG-MB2-C′ dots (ii). Inset shows photographs of the respective samples. (B) GPC elugrams of PEG-TMR-C′ dots (i) and MB2-PEG-TMR-C′ dots (ii). Inset shows photographs of the respective samples. Each sample pair was measured on the same day. Different sample pairs were measured on different days leading to differences in absolute peak elution times.

FIG. 9 depicts photosensitizing measurement. (A) Schematic representation of a photosensitizing measurement using 1,3-diphenylisobenzofuran (DPBF) as a singlet oxygen, ¹O₂, sensor. Absorption of a solution containing PEG-MB2-C′ dots and DPBF, measured at different time points, in the absence of 635 nm irradiation (dark toxicity). (B) Comparison of singlet oxygen, ¹O₂, generation from PEG-MB2-C′ dots and MB2-PEG-C′ dots in the dark.

FIG. 10 depicts GPC elugrams. (A) GPC elugrams of PEG-MB2-C′ dots (i) and c(RGDyC)-PEG-MB2-C′ dots (ii). Inset shows photographs of the respective samples. (B) GPC elugrams of MB2-PEG-C′ dots (i) and MB2-c(RGDyC)-PEG-C′ dots (ii). Inset shows photographs of the respective samples. Each sample pair was measured on the same day. Different sample pairs were measured on different days leading to differences in absolute peak elution times. (C) Absorption spectrum of c(RGDyC) in water, showing an isolated tyrosin peak (ε≈1400 M⁻¹ cm⁻¹).

### DETAILED DESCRIPTION OF THE DISCLOSURE

In the following description, reference is made to the accompanying drawings that form a part hereof, and in which is shown by way of illustration specific embodiments which may be practiced. These embodiments are described in detail to enable those skilled in the art to practice the disclosure, and it is to be understood that other embodiments may be utilized and that structural and logical changes may be made without departing from the scope of the present disclosure.

Although the present disclosure has been described for the purpose of illustration, it is understood that such detail is solely for that purpose and variations can be made by those skilled in the art without departing from the spirit and scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range. As an illustrative example, any range provided herein includes all values that fall within the ranges to the tenth decimal place, unless indicated otherwise.

As used herein, unless otherwise indicated, the term “group” refers to a chemical entity that is monovalent (i.e., has one terminus that can be covalently bonded to another chemical species), or divalent or polyvalent (i.e., has two or more termini that can be covalently bonded to other chemical species). A group may be referred to as a moiety. Illustrative examples of groups include, but are not limited to:

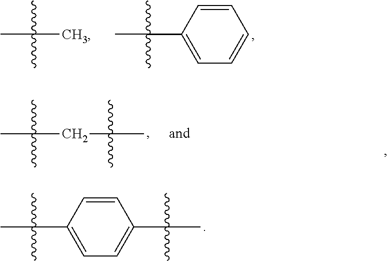

The present disclosure provides inorganic nanoparticles. The nanoparticles are also referred to herein as ultrasmall nanoparticles or C′ dots.

The inorganic nanoparticles, which may silica-based nanoparticles or aluminosilica-based nanoparticles with sizes below the critical size for renal clearance in the body (e.g., sizes below 10 nm diameter), may be used in photodynamic therapy (PDT). The ability to further surface functionalize such nanoparticles with specific surface functional groups, including, for example, fluorescent dyes, chelators for radio-isotopes enabling positron emission tomography (PET) as well as radiotherapy, targeting moieties including, for example, specific peptides, antibodies, antibody fragments, carbohydrates, lipids, nucleic acids, and the like for specific targeting of, for example, tumor cells, as well as chemotherapy drugs, provides new nanoparticle platforms for therapeutic applications, and, optionally, optical imaging. The nanoparticles further provide access to efficient PDT probes (e.g., with up to order of magnitude or more improvement in singlet oxygen quantum yields).

The present disclosure also provides methods of making and using the inorganic nanoparticles. The techniques disclosed herein provide an aqueous synthesis approach to ultrasmall functional PEGylated PS-containing inorganic nanoparticles with improved control in multiple aspects, including particle size, particle size distribution, composition, particle PEGylation, particle surface functionalization, synthesis yield, product purity and manufacture reliability. The systematic and precise control covering all these aspects in a single organic-inorganic hybrid nanomaterials synthesis system has never been achieved before, preventing the safe translation of organic-inorganic hybrid nanomaterials from the laboratory to the clinic. Therefore, the techniques disclosed herein provide access to, for example, well-defined and systematically highly tunable silica-based and aluminosilicate-based nanomaterials that show significant potential in nanomedicine applications.

In an aspect, the present disclosure provides inorganic nanoparticles. The nanoparticles may be silica nanoparticles and aluminosilicate nanoparticles. The nanoparticles comprise one or more photosensitizer. The photosensitizer(s) are covalently bound to the silica matrix or aluminosilicate matrix of the nanoparticle and/or covalently bound to an exterior surface of the nanoparticle and/or are part of a PEG group. The nanoparticles are microporous. The nanoparticles comprise a silica core or an aluminosilicate core and the silica core or an aluminosilicate core comprises one or more PEG groups and/or functionalized PEG groups covalently bound to a surface of the silica core or an aluminosilicate core. A nanoparticle may be present in a composition comprising a plurality of nanoparticles of the present disclosure.

Ultrasmall (sub-10 nm) organic-inorganic hybrid silica nanoparticles loaded with photosensitizer molecules (which may be in the form of photosensitizer groups), which may be referred to as silica nanophotosensitizers (SNPSs), and aluminosilicate nanoparticles loaded with photosensitizer molecules present a way to meet the unmet need in the art. In various examples, two different particle designs of ultrasmall poly(ethylene glycol) coated (PEGylated) SNPSs covalently binding the methylene blue derivative MB2 are described. In an approach (design one), MB2 is encapsulated into the silica matrix, while in another approach (design two), MB2 is grafted on the particle surface in between chains of the stabilizing PEG corona. Both cases were compared with regard to their singlet oxygen quantum yields, Φ_{Δ}, with the effective Φ_{Δ}^{eff} per particle reaching 111% and 161% for design one and two, respectively. Also, it was shown that both particle designs allow functionalization with a targeting peptide, c(RGDyC), rendering SNPSs a platform for medical applications.

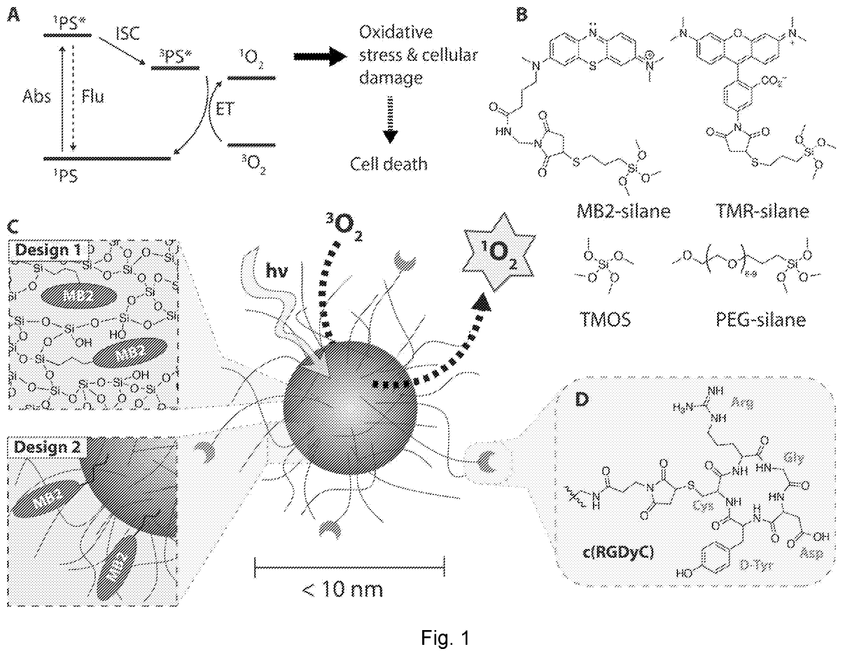

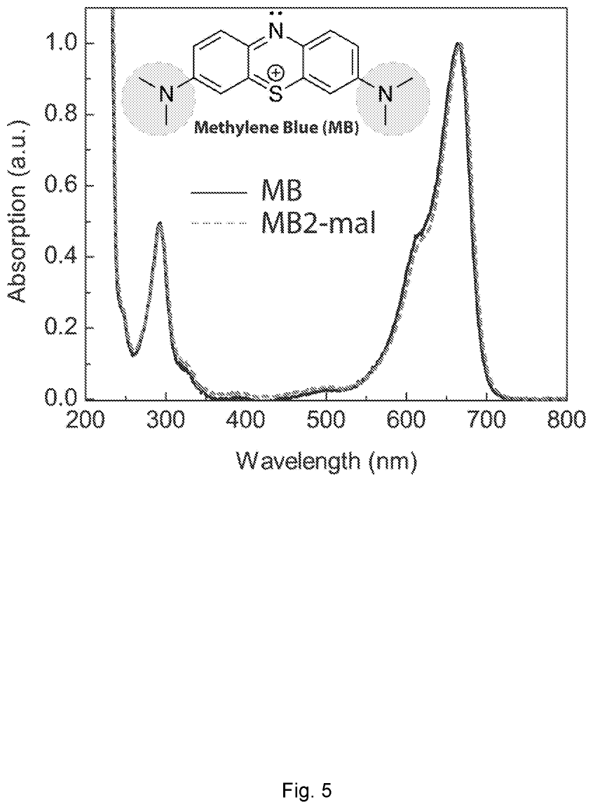

The chemical inertness, optical transparency of silica, and cost-effective water-based synthesis paired with exceptional size and structural control on the sub-nanometer length scale, insusceptibility to swelling due to pH changes, and high silica matrix porosity render such particles ideal candidates for PDT. The present disclosure describes, in certain examples, ultrasmall sub-10 nm organic-inorganic hybrid SNPs covalently binding the PS MB2, a derivate of methylene blue (MB) (FIG. 1B and FIG. 5). The nanoparticles may be referred to as silica nanophotosensitizers (SNPSs). MB is desirable due to its high singlet oxygen quantum yield and extinction coefficient in the near infrared (Φ_{Δ}≈0.5, ε=10⁵ M⁻¹ cm⁻¹ at 664 nm).

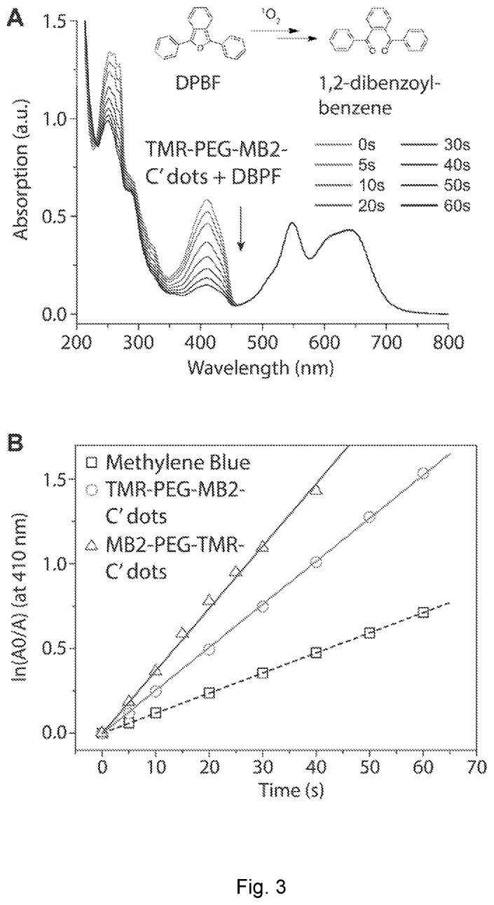

In examples, two different SNPS designs (FIG. 1C) are described. In design one, MB2 is encapsulated within the silica network of an ultrasmall poly(ethylene glycol) coated (PEGylated) SNP; in design two, MB2 is grafted onto the SNP surface, inserted between the PEG corona chains. In both cases the PS molecules are covalently bound to the SNPSs via a thiol-Michael addition click reaction between maleimide functionalized MB2 and (3-mercaptopropyl)trimethoxysilane (MPTMS). We show that both particle types can be further functionalized with the 43 integrin-targeting cyclic(arginine-glycine-aspartic acid-D-tyrosine-cysteine) peptide (cRGDyC, FIG. 1D). Photosensitizing action is successfully demonstrated using the singlet oxygen sensor 1,3-diphenylisobenzofuran (DPBF, inset FIG. 3A). We demonstrate effective per particle singlet oxygen quantum yields of 111% (design one) and 161% (design two), respectively.

Presented herein, as examples, are the synthesis of two different designs for ultrasmall organic-inorganic hybrid silica nanophotosenistizers, that covalently bind the methylene blue derivate MB2. It was found that the properties of MB2 strongly depend on the particle design. Both particle designs yielded sub-10 nm size particles that could be functionalized with c(RGDyC) as a targeting moiety. Despite reduced singlet oxygen quantum yields of MB2 upon particle association, the effective particle singlet oxygen quantum yields far exceed the quantum yield of a single MB2 PS. The advantages of ultrasmall organic-inorganic hybrid functionalized silica nanoparticles as a delivery and protective system for photosensitizers make such probes useful for applications in PDT. Photophysical considerations: A PS has unique photophysical characteristics. Generation of ¹O₂ is catalyzed by photoexcitation of the PS. FIG. 1A depicts a simplified Jablonski scheme illustrating the photophysical processes leading to ¹O₂ generation. From an electronically excited singlet state the PS undergoes a forbidden electron spin-flip (intersystem crossing, ISC) into an energetically lower lying excited triplet state, ³PS*. From here, ³PS* relaxes into the singlet ground state, ¹PS, via energy transfer (ET) with dissolved molecular triplet oxygen, ³O₂, yielding cytotoxic reactive singlet oxygen, ¹O₂. High intersystem crossing rates (k_{ISC}) and long triplet state lifetimes (τ_{T}>1 μs) of the PS promote ¹O₂ generation, which is reflected in high singlet oxygen quantum yields, Φ_{Δ}. A desirable PS should have a molar extinction coefficient of ε≥50 000 M⁻¹ cm⁻¹ in the therapeutic window of ˜600-1200 nm and a singlet oxygen quantum yield of Φ_{Δ}≥0.5. In addition, high photostability, as well as low phototoxicity in the dark are desired.

Chemical considerations: It is desirable that the nanoparticles for use as PDT probes be non-toxic. It may also be desirable that the probe be localized at a specific site of interest. Singlet oxygen is highly reactive, and locally produced by the PS. Typical diffusion lengths of singlet oxygen in tissue before it reacts are on the order of tens of nanometers. Therefore, to minimize damage of healthy tissue, selective targeting is important. Since certain PS molecules are hydrophobic and prone to aggregation in physiological environments, low selectivity towards diseased tissue and adverse pharmacokinetics have hindered their clinical translation. Use of the nanoparticles (NPs) as PS delivery vehicles may promote solubility, overcome aggregation issues to improve pharmacokinetics, and protect PSs from enzymatic degradation. Furthermore, NP surface functionalization with targeting moieties may reduce systemic side effects, increases the therapeutic concentration of PSs at the target site, and give room for multi-modality platforms simultaneously enabling diagnosis, imaging, and treatment.

Structural considerations: Since PSs are bound to NPs and do not have to be released, it is important that oxygen species can easily diffuse to and away from the PS molecule. After NPs have targeted the site of interest, in the case of NPs having targeting capability, and PDT has been performed (or if the NPs have failed to target the site of disease in the first place) it is desirable that they are rapidly cleared from the body to reduce potential side effects (principle of target-or-clear). Both of these considerations favor small hydrodynamic diameters leading to rapid renal clearance via the kidneys with a cutoff for NPs below 10 nm hydrodynamic diameter.

A nanoparticle can comprise various photosensitizers (PSs) (e.g., photosensitizer groups). An excited photosensitizer molecule undergoes a quantum mechanically forbidden electron spin-flip resulting in an energetically excited triplet state, ³PS*. From here, ³PS* relaxes into the singlet ground state, ¹PS, via energy transfer with, for example, dissolved molecular triplet oxygen, ³O₂, which may yield cytotoxic reactive singlet oxygen, ¹O₂. A photosensitizer may exhibit a triplet state lifetime of at least 0.5 microseconds, at least one microsecond, at least 2 microseconds, at least 3 microseconds, at least 4 microseconds, at least 5 microseconds, at least 10 microseconds, at least 25 microseconds, or at least 50 microseconds and/or a singlet oxygen quantum yield of at least 20%, at least 40%, at least 45%, at least 50%, or at least 55%. Methods of determining triplet state lifetime are known in the art. Triplet lifetime may be determined indirectly from time-resolved luminescence from singlet oxygen and/or directly from photo-induced absorption (PIA). Methods of determining singlet oxygen quantum yield are known in the art. Singlet oxygen quantum yield may be determined as described herein. A nanoparticle may comprise a mixture of photosensitizers. In various examples, a photosensitizer is a dye. In various examples, a photosensitizer is a NIR and/or IR photosensitizer. In various examples, a photosensitizer is a hydrophobic NIR and/or IR photosensitizers. Non-limiting examples of photosensitizers include psoralens (e.g., 5-methoxypsoralen and the like), porphyrinoids (e.g., porphyrins, chlorins, bacteriochlorins, phthalocyanines, and naphthalocyanines, and the like), phenothiazines (e.g., methylene blue (e.g., MB2), toluidine blue, and the like), cyanines (e.g., merocyanine 540 and the like), curcuminoids (e.g., curcumin and the like), BODIPY (e.g., BODIPY 650/665 and the like), xanthenes (e.g., Rose Bengal and the like), 4,5-dibromorhodamine methyl ester (TH9402), derivatives or analogs thereof, groups derived therefrom, and combinations thereof.

A nanoparticle comprises one or more groups derived from a PS molecule. A PS molecule may be hydrophobic. For example, a PS molecule or a derivative of a PS molecule is covalently bonded to the network of a nanoparticle (e.g., the silica network of a silica nanoparticle or the aluminosilicate network of an aluminosilicate nanoparticle) and, optionally, between two of the PEG groups). The resulting covalently bonded PS group is derived from an original PS molecule. Illustrative, non-limiting examples of groups derived from a PS molecule are described herein. In an example, a PS is incorporated into the silica or aluminosilicate network using a PS precursor that comprises a PS conjugated to a sol-gel silica precursor (e.g., a —Si(OR)₃ group, where R is an alkyl group).

The photosensitizers can be conjugated to a nanoparticle via various groups. The group conjugating a photosensitizers to a nanoparticle may be part of a photosensitizer precursor used in the synthesis of the nanoparticle. In various examples, the photosensitizers are conjugates via amino-silanes and active ester groups on the photosensitizers. In various examples, the photosensitizers are not conjugates via mercapto-silanes and maleimido groups on the photosensitizers.

A nanoparticle can have various amounts of photosensitizers. Without intending to be bound by any particular theory, it is considered that the number of photosensitizers present in a nanoparticle correlates to the amount of photosensitizers precursor used in the synthesis of the nanoparticle. As an illustrative example, for particles having a size below 10 nm, such particles (e.g., nanoparticle cores) may have, on average, 1 to 7 (e.g., 1-5) photosensitizers per nanoparticle (e.g., per nanoparticle core) and, optionally, additional photosensitizers covalently bound to a surface of the nanoparticle and/or part of a PEG group. In various examples, a nanoparticle (e.g., a nanoparticle core) comprises 1 or 2 photosensitizers and, optionally, additional photosensitizers covalently bound to a surface of the nanoparticle and/or part of a PEG group.

The number of photosensitizers per nanoparticle can be determined by methods known in the art. For example, the number of photosensitizer per nanoparticle is determined using a combination of fluorescence correlation spectroscopy (FCS), which provides the number of particle in solution (i.e. the particle concentration), and absorption spectroscopy on the particles, which provides the number of photosensitizers in the solution. Dividing the second number by the first gives you the number of photosensitizers per particle.

A nanoparticle can have various sizes. The size of a nanoparticle may be a longest dimension of the nanoparticle. The nanoparticle size may include the PEG corona (e.g., the PEG group(s)). For example, a nanoparticle has a size of 1-20 nm, including all 0.01 nm values and ranges therebetween (e.g., 1 to 20, 1 to 15 nm, 1-9.99 nm, 2 to 20, 2 to 15 nm, or 2-9.99 nm). In various examples, a nanoparticle has size of 1, 1.5, 2, 2.5, 3, 3.5, 4, 4.5, 5, 5.5, 6, 6.5, 7, 7.5, 8, 8.5, 9, 9.5, or 9.9 nm. In an example, a nanoparticle has a size of 2 to 7, 3 to 7, 4 to 7, 2 to 8, 3 to 8, 4 to 8, 5 to 7, or 6 to 7 nm. A size may be a hydrodynamic radius or hydrodynamic diameter. The size of a nanoparticle can be determined by methods known in the art. In various examples, nanoparticle size is determined by are determined by FCS and/or dynamic light scattering (DLS), or the like.

The silica core or aluminosilicate core can have various sizes. The size of a silica core or aluminosilicate core may be a longest dimension of the silica core or aluminosilicate core, respectively. The core size may not include the PEG corona (e.g., the PEG group(s)). For example, a nanoparticle core has a size of 2 to 15 nm, including all 0.01 nm values and ranges therebetween (e.g., 2-5 nm, 2 to 10 nm, 3 to 15 nm, or 2 to 9.99 nm). The size of a silica core or aluminosilicate core can be determined by methods known in the art. In various examples, silica core or aluminosilicate core size is determined by (are determined by) small-angle x-ray scattering (SAXS) and/or imaging techniques, such as, for example, transmission electron microscopy (TEM), or the like.

A nanoparticle or plurality of nanoparticles can exhibit desirable properties. For example, a nanoparticle or plurality of nanoparticles exhibit an increase of the singlet oxygen quantum yield, relative to the free photosensitizer(s) used in the nanoparticles in solution (e.g., aqueous solution), of 10% to 1000%, including all integer % values and ranges therebetween. In various examples, a nanoparticle or plurality of nanoparticles exhibit an increase of the singlet oxygen quantum yield, relative to the free photosensitizers(s) used in the nanoparticles in solution (e.g., aqueous solution), of 10% or more, 20% or more 30% or more, 40% or more, 50% or more, 75% or more, 100% or more, 250% or more, 500% or more, or 1000% or more.

A nanoparticle can have polyethylene glycol (PEG) group(s) disposed on (e.g., covalently bonded to) a surface of the nanoparticle. In an example, at least a portion of the exterior surface (e.g., at least 20%, 30%, 40% or 50% of the exterior surface) of a silica or aluminosilicate nanoparticle is functionalized with polyethylene glycol groups. In various examples, the number of PEG group(s) disposed on the surface of a nanoparticle is 3 to 600, including all integer number of PEG group(s) and ranges therebetween.

A silica nanoparticle or aluminosilicate nanoparticle may comprise a ligand or ligands disposed on (e.g., covalently bonded to) a surface of the nanoparticle. A nanoparticle may have two or more different ligands disposed on a surface. A ligand may be conjugated to (e.g., covalently bonded to) a surface of a nanoparticle. Suitable ligand conjugation methods are known in the art.

At least a portion of an exterior surface of a nanoparticle may be functionalized with at least one ligand. A nanoparticle can have various amounts of ligands. For example, a nanoparticle has 1-50 ligands disposed on (e.g., covalently bonded to) an exterior surface of the nanoparticle. In various examples, a nanoparticle has 1-3 ligands, 1-10 ligands, 1-20 ligands, or 1-40 ligands disposed on (e.g., covalently bonded to) an exterior surface of the nanoparticle.

The ligands carried by the nanoparticles include, but are not limited to, diagnostic and/or therapeutic agents (e.g., drugs). Examples of therapeutic agents include, but are not limited to, chemotherapeutic agents, antibiotics, antifungal agents, antiparasitic agents, antiviral agents, and combinations thereof. An affinity ligand may be also be conjugated to the nanoparticle to allow targeted delivery of the nanoparticles. For example, the nanoparticle may be conjugated to a ligand which is capable of binding to a cellular component (e.g., on the cell membrane or in the intracellular compartment) associated with a specific cell type. The targeted molecule may be a tumor marker or a molecule in a signaling pathway. The ligand can have specific binding affinity to certain cell types, such as, for example, tumor cells. In certain examples, the ligand may be used for guiding the nanoparticles to specific areas, such as, for example, liver, spleen, brain or the like. Imaging can be used to determine the location of the nanoparticles in an individual.

Examples of diagnostic agents include fluorescent dyes. Examples of fluorescent dyes and conjugation methods for fluorescent dyes are known in the art.

For example, a drug-linker conjugate, where the linker group can be specifically cleaved by enzyme or acid condition in tumor for drug release, may be covalently attached to the functional ligands on the particles for drug delivery. For example, drug-linker-thiol conjugates are attached to maleimido-PEG-particles through thiol-maleimido conjugation reaction post the synthesis of maleimido-PEG-particles. Additionally, both drug-linker conjugate and cancer targeting peptides may be attached to the particle surface for drug delivery specifically to tumor.

A ligand may be a biomolecule. Non-limiting examples of biomolecules include biotin, targeting ligands (e.g., targeting peptides, which may be natural or synthetic peptides, such as, for example, cyclic-RGD and derivatives thereof, alpha-MSH and derivatives thereof, and the like), targeting antibody or antibody fragments, targeting glycans (e.g., sugar molecules targeting cell surface receptors), nucleic acids (e.g., single stranded or double stranded DNA, various forms of RNA (e.g., siRNA, and the like), lipids, and carboyhydrates (e.g., oligosaccharides, polysaccharides, sugars, and the like). A ligand may be a chelator molecule for metal radioisotopes, such as, for example, deferoxamine (DFO), which is an efficient chelator for radio-labeling with, for example, Zr⁸⁹, NODA, DOTA, drug molecules, and the like. A chelator molecule can form a chelating moiety that binds a radio metal (e.g., radio label) to a nanoparticle. Nanoparticles with radio metals may be used to perform PET or radiotherapy. Nanoparticles with a drug molecule/molecules may be used in therapeutic methods.

In an aspect, the present disclosure provides a method of making nanoparticles. The methods are based on use of aqueous reaction medium (e.g., water). The nanoparticles can be surface functionalized with polyethylene glycol groups (e.g., PEGylated).

The methods as described herein may be linearly scaled up, e.g., from 10 ml reaction to 1000 ml or greater without any substantial change in product quality. This scalability is important for large scale manufacture of the nanoparticles.

The methods are carried out in an aqueous reaction medium (e.g., water). For example, the aqueous medium comprises water. Certain reactants are added to the various reaction mixtures as solutions in a polar aprotic solvent (e.g., DMSO or DMF). In various examples, the aqueous medium does not contain organic solvents (e.g., alcohols such as C₁ to C₆ alcohols) other than polar aprotic solvents at 10% or greater, 20% or greater, or 30% or greater. In an example, the aqueous medium does not contain alcohols at 1% or greater, 2% or greater, 3% or greater, 4% or greater, or 5% or greater. In an example, the aqueous medium does not contain any detectible alcohols. For example, the reaction media of any of the steps of any of the methods disclosed herein consists essentially of water and, optionally, a polar aprotic solvent.

At various points in the methods the pH may be adjusted to a desired value or within a desired range. The pH the reaction mixture can be increased by addition of a base. Examples of suitable bases include ammonium hydroxide.

For example, a method of making nanoparticles, which can be surface functionalized with polyethylene glycol groups (i.e., PEGylated) comprises: a) forming a reaction mixture at room temperature (e.g., 15° C. to 25° C. depending on the location) comprising water and TMOS (a silica core forming monomer) (e.g., at a concentration of 11 mM to 270 mM), wherein the pH of the reaction mixture (which can be adjusted using a base such as, for example, ammonium hydroxide) is 6 to 9 (which results in formation of core precursor nanoparticles having an average size (e.g., longest dimension) of, for example, 1 nm to 2 nm); b) holding the reaction mixture at a time (t¹) and temperature (T¹) (e.g., (t¹) 0.5 days to 7 days at room temperature to 95° C. (T¹)), whereby nanoparticles (core nanoparticles), which may have an average size (e.g., longest dimension) of 2 to 15 nm are formed; c) adjusting, if necessary, the pH of the reaction mixture to a pH of 6 to 10 comprising the core nanoparticles; and d) optionally (PEGylating the core nanoparticles by) adding at room temperature to the reaction mixture comprising the core nanoparticles, respectively, a PEG-silane conjugate (comprising a PEG moiety covalently bound to a silane moiety) (e.g., at a concentration of 10 mM to 60 mM) (e.g., PEG-silane conjugate dissolved in a polar aprotic solvent such as, for example, DMSO or DMF) and holding the resulting reaction mixture at a time (t²) and temperature (T²) (e.g., (t²) 0.5 minutes to 24 hours at room temperature (T²)) (whereby at least a portion of the PEG-silane conjugate molecules are adsorbed on at least a portion of the surface of the core nanoparticles from b)); e) heating the mixture from d) at a time (t³) and temperature (T³) (e.g., (t³) 1 hour to 24 hours at 40° C. to 100° C. (T³)), whereby the nanoparticles surface functionalized with polyethylene glycol groups are formed.

The nanoparticles may be subjected to post-synthesis processing steps. For example, after synthesis (e.g., after e) in the example above) the solution is cooled to room temperature and then transferred into a dialysis membrane tube (e.g., a dialysis membrane tube having a Molecular Weight Cut off 10,000, which are commercially available (e.g., from Pierce)). The solution in the dialysis tube is dialyzed in DI-water (volume of water is 200 times more than the reaction volume, e.g., 2000 ml water for a 10 ml reaction) and the water is changed every day for one to six days to wash away remaining reagents, e.g., ammonium hydroxide and free silane molecules. The particles are then filtered through a 200 nm syringe filter (fisher brand) to remove aggregates or dust. If desired, additional purification processes, including gel permeation chromatography and high-performance liquid chromatography, may be applied to the nanoparticles to further ensure the high purify of the synthesized particles (e.g., 1% or less unreacted reagents or aggregates). After any purification processes, the purified nanoparticles may be transferred back to deionized water if other solvent is used in the additional processes.

The cores may be silicon cores. The reaction mixture used in silicon core formation can comprise TMOS as the only silicon core forming monomer.

The cores may be aluminosilicate cores. The reaction mixture used in aluminosilicate core formation can comprise TMOS as the only silicon core forming monomer and one or more alumina core forming monomer (e.g., an aluminum alkoxide such as, for example, aluminum-tri-sec-butoxide or a combination of aluminum alkoxides).

In the case of aluminosilicate core synthesis, the pH of the reaction mixture is adjusted to a pH of 1 to 2 prior to addition of the alumina core forming monomer. After aluminosilicate core formation, the pH of the solution is adjusted to a pH of 7 to 9 and, optionally, PEG with molecular weight between 100 and 1,000 g/mol, including all integer values and ranges therebetween, at concentration of 10 mM to 75 mM, including all integer mM values and ranges therebetween, is added to the reaction mixture prior to adjusting the pH of the reaction mixture to a pH of 7 to 9.

The reaction mixture used to form nanoparticles comprises one or more photosensitizer precursor. In this case, the resulting nanoparticles have one or more photosensitizer molecules encapsulated and/or incorporated therein. For example, a core nanoparticle has 1, 2, 3, 4, 5, 6, or 7 photosensitizer molecules encapsulated therein. Mixtures of photosensitizer precursors may be used. The photosensitizer precursor is a photosensitizer conjugated to a silane. For example, a photosensitizer with maleimido functionality is conjugated to thiol-functionalized silane. In another example, a photosensitizer with NHS ester functionality is conjugated to amine-functionalized silane. Examples of suitable silanes and conjugation chemistries are known in the art. Examples of suitable photosensitizers, but are not limited to, psoralens (e.g., 5-methoxypsoralen and the like), porphyrinoids (e.g., porphyrins, chlorins, bacteriochlorins, phthalocyanines, and naphthalocyanines, and the like), phenothiazines (e.g., methylene blue (e.g., MB2), toluidine blue, and the like), cyanines (e.g., merocyanine 540 and the like), curcuminoids (e.g., curcumin and the like), BODIPY (e.g., BODIPY 650/665 and the like), xanthenes (e.g., Rose Bengal and the like), 4,5-dibromorhodamine methyl ester (TH9402), derivatives or analogs thereof, groups derived therefrom, and combinations thereof, and the nanoparticles surface functionalized with polyethylene glycol groups have one or more PS molecules encapsulated therein.

The photosensitizer(s) (e.g., photosensitizer group(s)) of the individual nanoparticles may be completely encapsulated within the individual nanoparticles, partially encapsulated within the individual nanoparticles, disposed on the surface of the individual nanoparticles, are part of a PEG group of the individual nanoparticles, or a combination thereof. All of the photosensitizer(s) (e.g., nanophotosensitizer group(s)) may only be encapsulated (e.g., covalently bound to the nanoparticle core matrix) by the nanoparticle core. In this case, the nanoparticles may not exhibit detectible surface presence of the photosensitizer(s) as determined by high-performance liquid chromatography (HPLC).

Inorganic nanoparticles may be analyzed by high performance liquid chromatography (HPLC). HPLC may be used to determine the location of one or more photosensitizer group.

A method of analyzing inorganic nanoparticles may comprise: depositing an inorganic nanoparticle in an HPLC column comprising an input in fluid communication with a stationary phase in fluid communication with an output in fluid communication with a detector; passing a mobile phase through the HPLC column, such that the inorganic nanoparticle elutes from the column and enters the detector, such that the detector generates a signal, wherein the signal indicates the location of the one or more PS group on and/or in the nanoparticle and/or core-shell nanoparticle; and analyzing the signal to determine the location of the one or more PS group on and/or in the inorganic nanoparticle. The signal comprises a retention time that correlates to the location of one or more PS group on and/or in (e.g., encapsulated by or partially encapsulated by) an inorganic nanoparticle. A peak at a specific retention time may also correlate to the number of PS groups disposed and/or partially disposed on the exterior surface of an inorganic particle or whether PS groups are in (e.g., encapsulated by or partially encapsulated by) an inorganic nanoparticle.

In an example, whenever eluent comprising inorganic nanoparticles passes through a detector, the detector generates a signal with an intensity greater than baseline. The relative time at which a signal occurs following the injection of a sample comprising a plurality of inorganic nanoparticles in the column determines the elution time of a portion of the plurality of inorganic nanoparticles. The elution time correlates to a portion of inorganic nanoparticles eluted from the column, with more hydrophobic particles being eluted at later times. Without intending to being bound by any particular theory, it is expected that an increasing number of photosensitizer groups, which may be hydrophobic photosensitizer groups, disposed on the surface of an inorganic nanoparticle increases the inorganic nanoparticle's elution time. As an illustrative example, a inorganic nanoparticle that has two photosensitizer groups disposed or partially disposed on the surface elutes later than an inorganic nanoparticle with only one PS disposed or partially disposed on the surface.

Various HPLC columns are suitable for a method of analyzing an inorganic nanoparticle via HPLC. An HPLC column may be a reverse-phase HPLC column (RP-HPLC column). An RP-HPLC column may comprise a C4 stationary phase or a C8 stationary phase or other suitable moderately hydrophilic stationary phases. An RP-HPLC column may have various lengths. For example, a suitable RP-HPLC column is 100 to 300 mm long, including every integer mm value and range therebetween (e.g., 150-250 mm in length, such as, for example, 150 mm in length). An RP-HPLC column may have various pore sizes. For example, a suitable RP-HPLC column has a pore size of 200 to 400, including every integer A value and range therebetween (e.g., 250 to 350, such as, for example, 300 Å). An RP-HPLC column may have various particle sizes. For example, a suitable RP-HPLC column has a particle size of 2 to 6 μm, including every 0.1 μm value and range therebetween (e.g., 3.5 to 5 μm). Various detectors are suitable for a method of analyzing an inorganic nanoparticle via HPLC. Examples of suitable detectors include, but are not limited to, a UV detector (e.g., a tunable UV detector), an evaporative light scattering detector, a charged aerosol detector, a fluorescence-based detector (e.g., a fluorimeter), a photodiode array detector, and the like, and combinations thereof.

Various mobile phases are suitable for a method of analyzing an inorganic nanoparticle via HPLC. A mobile phase is an aqueous mobile phase, such as, for example, a water and acetonitrile mixture or a water and isopropanol and/or methanol mixture. A mobile phase may further comprise an acid, such as, for example, trifluoroacetic acid (TFA) or formic acid at a concentration of 0.01 to 1% by volume. Other suitable mobile phases are known in the art. The mobile phase may be passed through the column in a step-like gradient.

The nanoparticles can be PEG functionalized by various methods. Methods of PEG functionalization are known in the art. Combinations of PEG functionalization methods may be used. The nanoparticles may be PEG functionalized by methods comprising post-PEGylation surface modification by insertion (PPSMI). Non-limiting methods of PEG functionalization are described in PCT Application No. PCT/US16/30752, filed on May 4, 2016, and published as WO 2016/179260 on Nov. 11, 2016, and U.S. patent application Ser. No. 15/571,420, filed on Nov. 2, 2017 and published as U.S. Pat. Appl. Publ. No. 2018-0133346 on May 17, 2018, the disclosures of which with respect to PEG functionalization are incorporated herein by reference.

After core nanoparticle formation, the core nanoparticles may be reacted with one or more PEG-silane conjugates. Various PEG-silane conjugates can be added together or in various orders. This process is also referred to herein as PEGylation. The conversion percentage of PEG-silane is between 5% and 40% and the polyethylene glycol surface density is 1.3 to 2.1 polyethylene glycol molecules per nm². The conversion percentage of ligand-functionalized PEG-silane is 40% to 100% and the number of ligand-functionalized PEG-silane precursors reacted with each particle is 3 to 90.

PEGylation can be carried out at a variety of times and temperatures. For example, in the case of silica core nanoparticles, PEGylation may be carried out by contacting the nanoparticles at room temperature for 0.5 minutes to 24 hours (e.g., overnight). For example, in the case of alumina-silicate nanoparticles (e.g., alumina-silicate core nanoparticles) the temperature is 80° C. overnight.

The chain length of the PEG moiety of the PEG-silane (i.e., the molecular weight of the PEG moiety) can be tuned from 3 to 24 ethylene glycol monomers (e.g., 3 to 6, 3 to 9, 6 to 9, 8 to 12, or 8 to 24 ethylene glycol monomers). The PEG chain length of PEG-silane may be selected to tune the thickness of the PEG layer surrounding the particle and the pharmaceutical kinetics profiles of the PEGylated particles. The PEG chain length of ligand-functionalized PEG-silane may be used to tune the accessibility of the ligand groups on the surface of the PEG layer of the particles resulting in varying binding and targeting performance.

PEG-silane conjugates can comprise a ligand. The ligand is covalently bound to the PEG moiety of the PEG-silane conjugates (e.g., via though the hydroxy terminus of the PEG-silane conjugates). The ligand may be conjugated to a terminus of the PEG moiety opposite the terminus conjugated to the silane moiety. The PEG-silane conjugate may be formed using a heterobifunctional PEG compound (e.g., maleimido-functionalized heterobifunctional PEGs, NHS ester-functionalized heterobifunctional PEGs, amine-functionalized heterobifunctional PEGs, thiol-functionalized heterobifunctional PEGs, etc.). Examples of suitable ligands include, but are not limited to, peptides (natural or synthetic), cyclic peptides (e.g., cyclic-RGD and derivatives thereof, alpha-MSH and derivatives thereof, and the like), nucleic acids (e.g., single stranded or double stranded DNA, various forms of RNA (e.g., siRNA, and the like), lipids, carboyhydrates (e.g., oligosaccharides, polysaccharides, sugars, and the like), ligands comprising a radio label (e.g., ¹²⁴I, ¹³¹I, ²²⁵Ac or ¹⁷⁷Lu, ⁸⁹Zr, ⁶⁴Cu, and the like), antibodies, antibody fragments, ligands comprising a reactive group (e.g., a reactive group that can be further conjugated, for example, via click chemistry, to a molecule such as, for example, a pharmaceutical product (e.g., a drug molecule, which may be a toxic drug molecule, a small molecule inhibitor (e.g., gefitinib, and the like)).

For example, amine- and/or thiol-functionalized silane molecules are inserted between PEG chains and onto the silica surface of nanoparticles (e.g., C′ dots), to which additional functional ligands can subsequently be attached. This post-PEGylation surface modification by insertion (PPSMI) approach only requires a few extra steps sandwiched between nanoparticle (e.g., C′ dot) PEGylation and purification in a one-pot type water-based synthesis without diminishing high quality NP generation. The resulting nanoparticles (e.g., C′ dots) with additional functionalities exhibit physico-chemical properties like their size and PEG density close to clinically translated nanoparticles (e.g., C dots), opening a gate to the diversification of their clinical applications. Modification of a nanoparticle synthesis (e.g., a C′ dot synthesis) enables, for example, large numbers of targeting peptides per particle, as well as a facile and versatile spectroscopic approach to quantitatively assess the specific numbers of the different surface ligands by deconvolution of absorption spectra into individual components.

For example, PEG-silane conjugate comprising a ligand is added in addition to PEG-silane (e.g., in d) in the example above). In this case, nanoparticles surface functionalized with polyethylene glycol groups and polyethylene groups comprising a ligand are formed. The conversion percentage of ligand-functionalized or reactive group-functionalized PEG-silane is 40% to 100% and the number of ligand-functionalized PEG-silane precursors reacted with each particle is 3 to 600.

For example, before or after (e.g., 20 seconds to 5 minutes before or after) the PEG-silane conjugate is added (e.g., in d) in the example above) a PEG-silane conjugate comprising a ligand (e.g., at concentration between 0.05 mM and 2.5 mM) is added at room temperature to the reaction mixture comprising the core nanoparticles (e.g., from b) in the example above). The resulting reaction mixture is held at a time (t⁴) and temperature (T⁴) (e.g., (t⁴) 0.5 minutes to 24 hours at room temperature (T⁴)), where at least a portion of the PEG-silane conjugate molecules are adsorbed on at least a portion of the surface of the core nanoparticles (e.g., from b) in the example above). Subsequently, the reaction mixture is heated at a time (t⁵) and temperature (T⁵) (e.g., (t⁵) 1 hour to 24 hours at 40° C. to 100° C. (T⁵)), where nanoparticles surface functionalized with polyethylene glycol groups comprising a ligand are formed. Optionally, subsequently adding at room temperature to the resulting reaction mixture comprising nanoparticles surface functionalized with polyethylene glycol groups comprising a ligand a PEG-silane conjugate (the concentration of PEG-silane no ligand is between 10 mM and 75 mM) (e.g., PEG-silane conjugate dissolved in a polar aprotic solvent such as, for example, DMSO or DMF), holding the resulting reaction mixture at a time (t⁶) and temperature (T⁶) (e.g., (t⁶) 0.5 minutes to 24 hours at room temperature (T⁶)) (whereby at least a portion of the PEG-silane conjugate molecules are adsorbed on at least a portion of the surface of the nanoparticles surface functionalized with polyethylene glycol groups comprising a ligand a PEG-silane conjugate, and heating the resulting mixture from at a time (t⁷) and temperature (T⁷) (e.g., (t⁷) 1 hour to 24 hours at 40° C. to 100° C. (T⁷)), whereby nanoparticles surface functionalized with polyethylene glycol groups and polyethylene glycol groups comprising a ligand are formed.

In another example, at least a portion of or all of the PEG-silane has a reactive group on a terminus of the PEG moiety opposite the terminus conjugated to the silane moiety of the PEG-silane conjugate (is formed from a heterobifunctional PEG compound) and after formation of the nanoparticles surface functionalized with polyethylene glycol groups having a reactive group, and, optionally, polyethylene glycol groups, are reacted with a second ligand (which can be the same or different than the ligand of the nanoparticles surface functionalized with polyethylene glycol groups and polyethylene glycol group comprising a ligand is functionalized with a second reactive group (which can be the same or different than the reactive group of the nanoparticles surface functionalized with polyethylene glycol groups and polyethylene glycol group comprising a ligand) thereby forming nanoparticles surface functionalized with polyethylene groups functionalized with a second ligand and, optionally, polyethylene glycol groups.

In another example, at least a portion of or all of the PEG-silane has a reactive group on a terminus of the PEG moiety opposite the terminus conjugated to the silane moiety of the PEG-silane conjugate (is formed from a heterobifunctional PEG compound) and after formation of the nanoparticles surface functionalized with polyethylene glycol groups and, optionally having a reactive group, and, optionally, polyethylene glycol groups, are reacted with a second ligand (which can be the same or different than the ligand of the nanoparticles surface functionalized with polyethylene glycol groups and polyethylene glycol group comprising a ligand) functionalized with a second reactive group (which can be the same or different than the reactive group of the nanoparticles surface functionalized with polyethylene glycol groups and polyethylene glycol group comprising a ligand) thereby forming nanoparticles surface functionalized with polyethylene groups functionalized with a second ligand and, optionally, polyethylene glycol groups, or where at least a portion of the PEG-silane has a reactive group on a terminus of the PEG moiety opposite the terminus conjugated to the silane moiety of the PEG-silane conjugate (is formed from a heterobifunctional PEG compound) and after formation of the nanoparticles surface functionalized with polyethylene glycol groups having a reactive group, nanoparticles surface functionalized with polyethylene glycol groups having a reactive group and polyethylene glycol groups comprising a ligand, the reactive group(s) are reacted with a second ligand functionalized with a reactive group (which can be the same or different than the ligand of the nanoparticles surface functionalized with polyethylene glycol groups and polyethylene glycol group comprising a ligand) thereby forming nanoparticles surface functionalized with polyethylene glycol groups and polyethylene groups functionalized with a second ligand or nanoparticles surface functionalized with polyethylene glycol groups comprising a ligand that is functionalized with the second ligand.

The nanoparticles with PEG groups functionalized with reactive groups may be further functionalized with one or more ligands. For example, a functionalized ligand is reacted with a reactive group of a PEG group. Examples of suitable reaction chemistries and conditions for post-nanoparticle synthesis functionalization are known in the art.

The nanoparticles may have a narrow size distribution. In various examples, the nanoparticle size distribution (before or after PEGylation), not including extraneous materials such as, for example, unreacted reagents, dust particles/aggregates, is +/−5, 10, 15, or 20% of the average particle size (e.g., longest dimension). The particle size can be determined by methods known in the art. For example, the particle size is determined by TEM, GPS, DLS, or a combination thereof. DLS contains systematic deviation and, therefore, the DLS size distribution may not correlate with the size distribution determined by TEM or GPS.

In an aspect, the present disclosure provides compositions comprising nanoparticles of the present disclosure. The compositions can comprise one or more types (e.g., having different average size and/or one or more different compositional feature) of nanoparticles. A composition may comprise a nanoparticle and a pharmaceutically acceptable carrier. The compositions, as synthesized and before any post-synthesis processing/treatment, may have nanoparticles, other particles (e.g., 2-15 nm), dust particles/aggregates (e.g., greater than 20 nm), unreacted reagents (e.g., less than 2 nm), or a combination thereof.

A composition may comprise a plurality of nanoparticles from a single reaction mixture or a plurality of nanoparticles from two or more different reaction mixtures. A composition may comprise nanoparticle having the same photosensitizer (incorporated in the same or different way) or a combination of two or more structurally distinct photosensitizers (each incorporated in the same or different way).

For example, a composition comprises a plurality of nanoparticles (e.g., silica nanoparticles, aluminosilicate nanoparticles, or a combination thereof). Any of the nanoparticles may be surface functionalized with one or more type of polyethylene glycol groups (e.g., polyethylene glycol groups, functionalized (e.g., functionalized with one or more ligand and/or a reactive group) polyethylene glycol groups, or a combination thereof). Any of the nanoparticles can have a PS or combination of PS encapsulated therein. The PS molecules are covalently bound to the nanoparticles. A composition or the nanoparticles comprising a composition may be made by a method of the present disclosure.

The nanoparticles in a composition can have a variety of sizes. The size of an individual nanoparticle in a composition may be a longest dimension of the nanoparticle. The size of an individual nanoparticle in a composition may include the PEG corona (e.g., the PEG group(s)). For example, the nanoparticles have a size of 1-20 nm, including all 0.01 nm values and ranges therebetween (e.g., 1 to 20, 1 to 15 nm, 1-9.99 nm, 2 to 20, 2 to 15 nm, or 2-9.99 nm). In various examples, the nanoparticles have a size of 1, 1.5, 2, 2.5, 3, 3.5, 4, 4.5, 5, 5.5, 6, 6.5, 7, 7.5, 8, 8.5, 9, 9.5, 9.9 nm, or combinations thereof. In an example, the nanoparticles have a size of 2 to 7, 3 to 7, 4 to 7, 2 to 8, 3 to 8, 4 to 8, 5 to 7, or 6 to 7 nm. A size may be a hydrodynamic radius or hydrodynamic diameter. The size of the nanoparticles can be determined by methods known in the art. In various examples, the size (e.g., size distribution) of nanoparticles in a composition is determined by are determined by FCS and/or dynamic light scattering (DLS), or the like.

The nanoparticles in a composition can have a variety of core (e.g., silica core or aluminosilicate core) sizes. The size of an individual silica core or aluminosilicate core may be a longest dimension of the silica core or aluminosilicate core, respectively. The core size may not include the PEG corona (e.g., the PEG group(s)). The nanoparticles may have a core size of 2 to 15 nm, including all 0.1 nm values and ranges therebetween (e.g., 2 to 5 nm, 2 to 10 nm, 3 to 15 nm, or 2 to 9.99). In various examples, the nanoparticles have a core size of 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15 nm, or a combination thereof. The size of the silica cores or aluminosilicate cores can be determined by methods known in the art. In various examples, silica core or aluminosilicate core size (or the size (e.g., size distribution) of nanoparticles in a composition) is determined by (are determined by) small-angle x-ray scattering (SAXS) and/or imaging techniques, such as, for example, transmission electron microscopy (TEM), or the like.

In various examples, at least 90%, 95%, 96%, 97%, 98%, 99%, 99.5% 99.9%, or 100% of the nanoparticles and/or nanoparticle cores in a composition have a size (e.g., longest dimension) of 1 to 9.99 nm, including all 0.1 nm values and ranges therebetween (e.g., 2 to 15 nm or 2 to 9.99), or 2 to 10 nm. In another example, at least 90%, 95%, 96%, 97%, 98%, 99%, 99.5%, or 99.9% or 100% of the nanoparticles and/or nanoparticle cores in a composition have a size (e.g., longest dimension) within (+/−) 20%, 15%, 10%, or 5% of the average size (e.g., average longest dimension) and the average size (e.g., average longest dimension) is 1 to 9.99 nm, including all 0.1 nm values and ranges therebetween (e.g., 2 to 15 nm or 2 to 9.99), or 2 to 10 nm. For a size distribution, the composition may not be subjected to any particle-size discriminating (particle size selection/removal) processes (e.g., filtration, dialysis, chromatography (e.g., GPC), centrifugation, etc.). For example, the nanoparticles of the present disclosure are the only nanoparticles in the composition.

The composition can comprise additional components. For example, the composition can also comprise a buffer suitable for administration to an individual (e.g., a mammal such as, for example, a human, or a non-human mammal). The buffer may be a pharmaceutically-acceptable carrier.

Pharmaceutically acceptable carriers are generally aqueous based. Some examples of materials which can be used in pharmaceutically-acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. (See REMINGTON'S PHARM. SCI., 15th Ed. (Mack Publ. Co., Easton (1975)).

In various aspects, the present disclosure provides uses of nanoparticles and/or compositions of the present disclosure. Non-limiting examples of uses of the nanoparticles and/or compositions of the present disclosure include imaging methods and photodynamic therapy (PDT) methods, and the like.

Nanoparticle(s) or composition(s) of the present disclosure can be used in various PDT methods. In various examples, a photodynamic therapy method (e.g., a method for treating cancer in an individual) comprises: administering to an individual (e.g., an individual with abnormal cells such as, for example, cancer cells) with a nanoparticle of the present disclosure or a composition of the present disclosure; and irradiating the individual (e.g., the abnormal cells of the individual and, optionally, the surrounding tissue) or a portion thereof (e.g., directing electromagnetic radiation into the individual (e.g., the abnormal cells of the individual and, optionally, the surrounding tissue) or a portion thereof) with electromagnetic radiation having a wavelength of 400 to 900 or 400 to 800 nm (e.g., one or more wavelengths that form at least one triplet state nanoparticle—PS of the nanoparticle—that can form a reactive ion species, for example, on energy transfer to an oxygen molecule or other oxygen containing species), wherein the irradiation results in formation of a reactive ion species (e.g., singlet oxygen) that inhibit the growth of and/or kill at least a portion of or all of the abnormal cells.

A PDT method may further comprise visualization of the cancer after administration of the nanoparticle or the composition. In an example, the visualization is carried out using fluorescence imaging.

Compositions comprising the present nanoparticles can be administered to an individual by any suitable route—either alone or as in combination with other agents. Administration can be accomplished by any means, such as, for example, by parenteral, mucosal, pulmonary, topical, catheter-based, or oral means of delivery. Parenteral delivery can include, for example, subcutaneous, intravenous, intramuscular, intra-arterial, and injection into the tissue of an organ. Mucosal delivery can include, for example, intranasal delivery. Pulmonary delivery can include inhalation of the agent. Catheter-based delivery can include delivery by iontophoretic catheter-based delivery. Oral delivery can include delivery of an enteric coated pill, or administration of a liquid by mouth. Transdermal delivery can include delivery via the use of dermal patches.

Following administration of a composition comprising the present nanoparticles (e.g., nanoparticles comprising a fluorescent group), the path, location, and clearance of the NPs may be monitored using one or more imaging techniques. Examples of suitable imaging techniques include Artemis Fluorescence Camera System.

In certain cases, combination therapy (PDT+chemotherapy) may reduce symptoms and prolong the life of patients significantly. This approach may be useful in treating patients with advanced cancers that are not suitable for surgery radiation therapy (e.g., patients with small cell lung cancer, bladder cancer, brain cancer, head and/or neck cancer esophageal cancer that cannot be completely removed by surgery).

In various examples, a method further comprises administering to the patient an additional cancer treatment. In some examples, the additional cancer treatment is chosen from the group comprising surgery, radiotherapy, chemotherapy, toxin therapy, immunotherapy, cryotherapy, gene therapy, and combinations thereof.

In an example, a PDT method further comprises administration of a chemotherapy agent. In various examples, a chemotherapy agent is a drug or drug formulation. Non-limiting examples of drug formulations include polymeric micelle formulations, liposomal formulations, dendrimer formulations, polymer-based nanoparticle formulations, silica-based nanoparticle formulations, nanoscale coordination polymer formulations, nanoscale metal-organic framework formulations, inorganic nanoparticle formulations, and the like.

Various chemotherapy agents (e.g., chemotherapy drugs) can be used. Any FDA approved chemotherapy agent (e.g., chemotherapy drugs) can be used. Combinations of chemotherapy agents may be used.

The administrations and irradiation can be carried out in various ways and in various orders. Typically, administration(s) of the nanoparticle(s) or composition(s) is/are carried out first, and, subsequently, the chemotherapy agent(s) is/are is administered. The irradiation is carried out after administration of the nanoparticle(s) or composition(s) and before administration of the chemotherapy agent(s) or after administration of both the nanoparticle(s) or composition(s) and chemotherapy agent(s). In an example, the administration comprises i) administration of the nanoparticle(s) and/or composition(s), and ii) after completion of the administration of the nanoparticle(s) and/or composition(s) and irradiation of the individual, administration of the chemotherapy agent.

In an example, the chemotherapy agent is administered (e.g., administration initiated) after administration (e.g., first administration) of the nanoparticle(s) or composition(s) or after administration (e.g., first administration) of the nanoparticle(s) or composition(s) and irradiation.

Without intending to be bound by any particular theory, it is considered that the irradiation causes a response (e.g., photodynamic therapy response) in the individual. “Irradiating” and “irradiation” as used herein includes exposing an individual to a selected wavelength or wavelengths of light. It is desirable that the irradiating wavelength is selected to match the wavelength(s) that excite the nanoparticle(s) (e.g., nanoparticle(s) of the composition(s). It is desirable that the radiation wavelength(s) matches the excitation wavelength(s) of the nanoparticle(s) and has low absorption by the non-target tissues of the individual.

Irradiation is further defined herein by its coherence (laser) or non-coherence (non-laser), as well as intensity, duration, and timing with respect to dosing using the nanoparticle(s) or composition(s) of the present disclosure. The intensity or fluence rate must be sufficient for the light to reach the target tissue. The duration or total fluence dose must be sufficient to photoactivate enough of the nanoparticle(s) or composition(s) to act on the target tissue. Timing with respect to dosing of the nanoparticle(s) or composition(s) may be important, because 1) the administered the nanoparticle(s) or composition(s) may require time to home in on target tissue and 2) the blood level of the nanoparticle(s) or composition(s) may decrease with time. The radiation energy is provided by an energy source, such as a laser or cold cathode light source, that is external to the individual, or that is implanted in the individual, or that is introduced into an individual, such as by a catheter, optical fiber or by ingesting the light source in capsule or pill form (e.g., as disclosed in. U.S. Pat. No. 6,273,904 (2001), the disclosure of which with regard to radiation energy is incorporated herein by reference).

A method of the present disclosure can be used to treat an individual with (e.g., diagnosed with) cancer. The treatment can have various results. In various examples, a method of the present disclosure results in at least one or more of the following: complete cure of the individual, remission, increased long-term survival of the individual, or reduced tumor volume.

Methods of the present disclosure can be used on various individuals. In various examples, an individual is a human or non-human mammal. Examples of non-human mammals include, but are not limited to, farm animals, such as, for example, cows, hogs, sheep, and the like, as well as pet or sport animals such as horses, dogs, cats, and the like. Additional non-limiting examples of individuals include rabbits, rats, mice, and the like. The nanoparticles or compositions comprising nanoparticles may be administered to individuals for example, in pharmaceutically-acceptable carriers, which facilitate transporting the nanoparticles from one organ or portion of the body to another organ or portion of the body.

A method may also comprise visualization of the abnormal cells (e.g., cancer cells) (e.g., visualization of one or more tumors) after administration of the nanoparticle(s) or composition(s) of the present disclosure. The visualization (e.g., fluorescence imaging) can be used to determine personalized treatment for an individual. For example, visualization is carried out using fluorescence imaging (e.g., fluorescence imaging of the present disclosure). A method may further comprise further comprise surgical intervention (e.g., surgical removal of at least a portion of or all of a cancerous tissue from the individual). The surgical removal may be guided by the visualization (e.g., fluorescence imaging).

For example, a PDT method further comprises imaging of a region within an individual comprises: administering to the individual nanoparticles or a composition of the present disclosure comprising one or more fluorescent dye molecules (e.g., fluorescent dye group(s)); directing excitation light into the subject, thereby exciting at least one of the one or more fluorescent dye molecules (e.g., fluorescent dye group(s)); detecting excited light, the detected light having been emitted by the fluorescent dye molecule(s) (e.g., fluorescent dye group(s)) in the individuals as a result of excitation by the excitation light; and processing signals corresponding to the detected light to provide one or more images (e.g., a real-time video stream) of the region within the subject.

Additionally or alternatively, radioisotopes may be further attached to the ligand groups (e.g., tyrosine residue or chelator) of the ligand-functionalized particles or to the silica matrix of the PEGylated particles without specific ligand functionalization for photoinduced electron transfer imaging. If the radioisotopes are chosen to be therapeutic, such as, for example, ¹²⁴I, ¹³¹I, ²²⁵Ac or ¹⁷⁷Lu, ⁸⁹Zr, ⁶⁴Cu, and the like, this in turn would result in particles with additional radiotherapeutic properties.

A fluorescent image can be obtained in various ways. For example, obtaining a fluorescence image comprises: detecting excited electromagnetic radiation, the detected electromagnetic radiation having been emitted by the fluorescent dye molecules (e.g., fluorescent dye group(s)) in the individual as a result of excitation by the excitation electromagnetic radiation; and processing signals corresponding to the detected electromagnetic radiation to provide one or more images of the region within the individual.

In PDT methods, it is desirable to use nanoparticle(s) or composition(s) with non-fluorescent dye(s) (e.g., photosensitizer(s)) that on irradiation (e.g., excitation) populate triplet states, which in turn will lead to triplet to singlet transitions in oxygen, which then kills cells.

Methods of the present disclosure can be used to treat various cancers (e.g., a tumor or tumors related to a cancer). Non-limiting examples of cancers include lung cancer, colon cancer, melanoma, head and/or neck cancer, esophageal cancer, laryngeal cancer, breast cancer, pancreatic cancer, renal cancer, bladder cancer, ovarian cancer, prostate cancer, testicular cancer, and the like, and combinations thereof.

In an aspect, the present disclosure provides kits. A kit comprises one of more nanoparticle and/or one or more composition of the present disclosure. The composition(s) may be pharmaceutical compositions.

In an example, a kit comprises one or more nanoparticle of the present disclosure and/or one or more composition of the present disclosure, and instructions for use of the nanoparticle(s) and/or composition(s) for treatment of (e.g., administration to) an individual.

In an example, a kit is or comprises a closed or sealed package that contains the nanoparticle(s) and/or composition(s). In certain examples, the package comprises one or more closed or sealed vials, bottles, blister (bubble) packs, or any other suitable packaging for the sale, or distribution, or use of the nanoparticle(s) and/or composition(s). The printed material can include printed information. The printed information may be provided on a label, or on a paper insert, or printed on the packaging material itself. The printed information can include information that identifies the compound in the package, the amounts and types of other active and/or inactive ingredients, and instructions for taking the composition, such as the number of doses to take over a given period of time, and/or information directed to a pharmacist and/or another health care provider, such as a physician, or a patient. The printed material can include an indication that the pharmaceutical composition and/or any other agent provided with it is for treatment of cancer and/or any disorder associated with cancer. In examples, the kit includes a label describing the contents of the container and providing indications and/or instructions regarding use of the contents of the kit to treat any cancer.

The steps of the methods described in the various embodiments and examples disclosed herein are sufficient to carry out the methods and produce the compositions of the present disclosure. Thus, in an embodiment, a method consists essentially of a combination of the steps of the methods disclosed herein. In another embodiment, a method consists of such steps.

Although one embodiment of this work focuses on the photosensitizer MB2, described design principles and synthesis methods are applicable to other photosensitizers, including hydrophobic NIR and IR photosensitizers with large singlet oxygen quantum yields. Non-limiting examples of photosensitizers include porphyrins, chlorins, phthalocyanines, naphthalocyanines, bacteriochlorins, and boron-dipyrromethene (BODIPY) (e.g., 4,4-difluoro-4-bora-3a,4a-diaza-s-indacene and the like) dyes. In an example, a photosensitizer is not a fluorescent dye. In an example, a photosensitizer is a therapeutic agent not a diagnostic agent. PEGylated silica can provide a water-soluble carrier for these cargos, to allow specific targeting and achieve high local concentrations at targeted sites, while avoiding aggregation in aqueous media.

A nanoparticle (e.g., a 10 nm or smaller silica nanoparticle) may comprise a photosensitizer (e.g., methylene blue, MB2, or derivative thereof) and, optionally, a targeting ligand (e.g., a targeting peptide, such as, for example c(RGDyC)). The nanoparticle may be coated (e.g., at least partially coated) with polyethylene glycol (PEG) groups. The photosensitizer (e.g., methylene blue, MB2, or derivative thereof) may be encapsulated in the nanoparticle (e.g., the silica matrix of the silica nanoparticle) and/or the photosensitizer (e.g., methylene blue, MB2, or derivative thereof) is grafted onto the surface (e.g., outer surface) of the nanoparticle between PEG groups. A nanoparticle may be present in a composition. A method treating an individual in need of treatment (e.g., an individual in need of treatment for cancer) may comprise administering a plurality of nanoparticles of the present disclosure or a composition of the present disclosure. The method may comprise: i) administering the plurality of nanoparticles of the present disclosure or a composition of the present disclosure to an individual in need of treatment; and ii) exposing (e.g., irradiating) the individual or a portion thereof to light (e.g., light having a wavelength of 400-900 nm, including every nm value and range therebetween). The method may further comprises administering a chemotherapy agent.

In the following Statements, various examples of the methods and compositions of the present disclosure are described:

Statement 1. A composition comprising a plurality of silica nanoparticles and/or aluminosilicate nanoparticles of the present disclosure (e.g., where the individual nanoparticles comprise silica cores or aluminosilicate cores and the silica cores or aluminosilicate cores are surface functionalized with polyethylene glycol (PEG) groups and comprise at least one photosensitizer group, and the nanoparticles have a size (e.g., longest dimension) of 1-20 nm, including all 0.01 nm values and ranges therebetween (e.g., 1 to 20, 1 to 15 nm, 1-9.99 nm, 2 to 20, 2 to 15 nm, or 2-9.99 nm), and/or at least 90%, 95%, 96%, 97%, 98%, 99%, 99.5%, or 99.9% or 100% of the nanoparticles have a size (e.g., longest dimension) of 1 to 9.99 nm) e.g., 2 to 15 nm or 2 to 9.99), or 2 to 10 nm, and/or the nanoparticles may have a core size of 2 to 15 nm, including all 0.1 nm values and ranges therebetween (e.g., 2 to 5 nm, 2 to 10 nm, 3 to 15 nm, or 2 to 9.99), and optionally, the composition have not be subjected to any particle-size discriminating (particle size selection/removal) processes (e.g., filtration, dialysis, chromatography (e.g., GPC), centrifugation, etc.).
Statement 2. A composition according to Statement 1, where all of the at least one photosensitizer groups are the same.
Statement 3. A composition according to Statement 1 or 2, where the individual nanoparticles comprise two or more photosensitizer groups and the at least two of the photosensitizers groups are structurally different photosensitizer groups.
Statement 4. A composition according to any one of the preceding Statements, where the individual nanoparticle cores comprise 1 to 7 (e.g., 1, 2, 3, 4, 5, 6, 7, or a combination thereof) photosensitizer groups.
Statement 5. A composition according to any one of the preceding Statements, where the individual nanoparticle cores comprise 1 to 7 (e.g., 1, 2, 3, 4, 5, 6, 7, or a combination thereof) photosensitizer groups and/or the nanoparticles have 1 to 30 photosensitizers covalently bound to a surface of the nanoparticle core and/or photosensitizers that are part of a PEG group.
Statement 6. A composition according to any one of the preceding Statements, where the at least one photosensitizer group (e.g., a NIR and/or IR photosensitizer group) is chosen from psoralen groups (e.g., 5-methoxypsoralen groups and the like), porphyrinoid groups (e.g., porphyrin groups, chlorin groups, bacteriochlorin groups, phthalocyanine groups, and naphthalocyanine groups, and the like), phenothiazine groups (e.g., methylene blue group (e.g., MB2 group), toluidine blue groups, and the like), cyanine groups (e.g., merocyanine 540 group, and the like), curcuminoid groups (e.g., curcumin groups and the like), BODIPY groups (e.g., BODIPY 650/665 groups and the like), xanthene groups (e.g., Rose Bengal group and the like), 4,5-dibromorhodamine methyl ester groups (TH9402 groups), derivatives or analogs thereof, and combinations thereof.
Statement 7. A composition according to any one of the preceding Statements, where the photosensitizer is covalently attached to the silica or aluminosilicate core matrix via a functional group such as, for example, a thioether linkage (e.g., formed using a functional silane (e.g., a mercapto-propyl-silane and the like)).
Statement 8. A composition according to any one of the preceding Statements, where the individual nanoparticles have at least one photosensitizer group completely or partially encapsulated within the nanoparticle.
Statement 9. A composition according to any one of the preceding Statements, where the at least one photosensitizer group is disposed on the surface of the nanoparticle or is part of a PEG group.
Statement 10. A composition according to any one of the preceding Statements, where the photosensitizer group(s) of the individual nanoparticles is/are completely encapsulated within the individual nanoparticles, partially encapsulated within the individual nanoparticles, disposed on the surface of the individual nanoparticles, are part of a PEG group of the individual nanoparticles, or a combination thereof.
Statement 11. A composition according to any one of the preceding Statements, where all of the photosensitizer group(s) of the individual nanoparticles is/are completely encapsulated within the individual nanoparticles (e.g., individual nanoparticle cores).
Statement 12. A composition according to any one of the preceding Statements, where the nanoparticles do not exhibit detectible surface presence of the photosensitizer(s) as determined by high-performance liquid chromatography (HPLC).
Statement 13. A composition according to any one of the preceding Statements, where at least a portion or all of the nanoparticles further comprise one or more functional group described herein (e.g., chosen from fluorescent dyes, chelators for radio-isotopes, biomolecules, targeting groups (e.g., targeting peptides, which may be natural or synthetic peptides, such as, for example, cyclic-RGD and derivatives thereof, alpha-MSH and derivatives thereof, and the like), targeting antibody or antibody fragments, targeting glycans (e.g., sugar molecules targeting cell surface receptors), nucleic acids (e.g., single stranded or double stranded DNA, various forms of RNA (e.g., siRNA, and the like), lipids, and carboyhydrates (e.g., oligosaccharides, polysaccharides, sugars, and the like), drugs, and combinations thereof), where the one or more functional group is covalently bound to a surface of the nanoparticle(s), part of a PEG group, or a combination thereof).
Statement 14. A composition according to any one of the preceding Statements, where at least a portion or all of the PEG groups of a portion or all of the nanoparticles comprise one or more ligand group described herein, where the ligand group is disposed on a surface of the nanoparticles and/or is part of a PEG group.
Statement 15. A composition according to Statement 14, further comprising one or more radioisotope (e.g., a radioisotope attached to the ligand group(s) (e.g., tyrosine residue or chelator of the ligand-functionalized particles or to the silica matrix of the PEGylated particles, which may be without specific ligand functionalization) (e.g., ¹²⁴I, ¹³¹I, ²²⁵Ac or ¹⁷⁷Lu, ⁸⁹Zr, ⁶⁴Cu, and the like), which may be used for photoinduced electron transfer imaging).
Statement 16. A composition according to Statement 14 or 15, where the radioisotopes are therapeutic radioisotopes, thereby forming nanoparticles with radiotherapeutic properties.
Statement 17. A composition according to any one of Statements 14-16, where the composition comprises a drug-linker conjugate covalently attached to at least a portion of the nanoparticles (e.g., covalently attached to one or more ligand group of the nanoparticles and/or part of a PEG group), where the nanoparticles may be used for drug delivery, and where the linker group may be configured to be cleaved by an enzyme or acidic environment in a tumor for drug release.
Statement 18. A composition according to any one of Statements 14-17, where the composition comprises a targeting group described herein covalently attached to at least a portion of the nanoparticles (e.g., covalently attached to one or more ligand group of the nanoparticles and/or part of a PEG group).
Statement 19. A composition according to any one of the preceding Statements, further comprising a pharmaceutically acceptable carrier (e.g., a pharmaceutically acceptable buffer).
Statement 20. A composition according to any one of the preceding Statements, where the composition has not been subjected to any particle-size discriminating (particle size selection/removal) process or processes (e.g., filtration, dialysis, or centrifugation).
Statement 21. A method of treating an individual in need of treatment (e.g., an individual in need of treatment for cancer), comprising administering one or more nanoparticle of the present disclosure and/or a composition of the present disclosure (e.g., a composition according to any one of Statements 1-20).
Statement 22. A method according to Statement 21, further comprising exposing (e.g., irradiating) the individual or a portion thereof to light (e.g., light having a wavelength of 400-900 nm, including every nm value and range therebetween).
Statement 23. A method according to Statement 21 or 22, where the nanoparticles comprise a drug and the drug is released in the individual.
Statement 24. A method according to Statement 23, where the drug is released in a selected portion of the individual.
Statement 25. A method according to any one of Statements 21-24, further comprising imaging the individual.
Statement 26. A method according to Statement 25, where the imaging is fluorescence imaging.
Statement 27. A method of making nanoparticles of the present disclosure (e.g., where the individual nanoparticles comprise silica cores or aluminosilicate cores and the silica cores or aluminosilicate cores are surface functionalized with polyethylene glycol (PEG) groups (i.e., PEGylated) and comprise at least one photosensitizer group, and the nanoparticles have a size (e.g., longest dimension) of 1-20 nm, including all 0.01 nm values and ranges therebetween (e.g., 1 to 20, 1 to 15 nm, 1-9.99 nm, 2 to 20, 2 to 15 nm, or 2-9.99 nm), and/or at least 90%, 95%, 96%, 97%, 98%, 99%, 99.5%, or 99.9% or 100% of the nanoparticles have a size (e.g., longest dimension) of 1 to 9.99 nm) e.g., 2 to 15 nm or 2 to 9.99), or 2 to 10 nm, and/or the nanoparticles may have a core size of 2 to 15 nm, including all 0.1 nm values and ranges therebetween (e.g., 2 to 5 nm, 2 to 10 nm, 3 to 15 nm, or 2 to 9.99), comprising: a) forming a reaction mixture at room temperature (e.g., 15° C. to 25° C. depending on the location) comprising: water, TMOS (e.g., at a concentration of 11 mM to 270 mM), and a photosensitizer precursor (e.g., a silane-photosensitizer conjugate such as, for example, a silane-MB2 conjugate), where the pH of the reaction mixture (which can be adjusted using a base such as, for example, ammonium hydroxide) is 6 to 9 (which results in formation of precursor nanoparticles having an average size (e.g., longest dimension) of, for example, 1 nm to 2 nm); b) holding the reaction mixture at a time (t¹) and temperature (T¹) (e.g., (t¹) 0.1 hour to 7 days at room temperature to 95° C. (T¹), such as 10-15 minutes), whereby the nanoparticles are formed, c) adjusting, if necessary, the pH of the reaction mixture to a pH of 6 to 10 comprising the nanoparticles from b), d) (PEGylating the nanoparticles (e.g., core nanoparticles) by) adding at room temperature to the reaction mixture comprising the nanoparticles (e.g., core nanoparticles) from b), a PEG-silane conjugate (comprising a PEG moiety covalently bound to a silane moiety) (e.g., at a concentration of 10 mM to 60 mM) (e.g., if necessary, PEG-silane conjugate dissolved in a polar aprotic solvent such as, for example, DMSO or DMF) and holding the resulting reaction mixture at a time (t²) and temperature (T²) (e.g., (t²) 0.5 minutes to 24 hours at room temperature (T²)) (whereby at least a portion of the PEG-silane conjugate molecules are adsorbed on at least a portion of the surface of the nanoparticles (e.g., core nanoparticles) from b)); and e) optionally heating the mixture from d) at a time (t³) and temperature (T³) (e.g., (t³) 1 hour to 24 hours at 40° C. to 100° C. (T³)), whereby the nanoparticles (e.g., core nanoparticles) surface functionalized with PEG groups are formed.
Statement 28. A method according to Statement 27, where the reaction mixture further comprises an alumina forming monomer (e.g., aluminum alkoxides such as, for example, aluminum-tri-sec-butoxide) and the pH of the reaction mixture is adjusted to a pH of 1 to 2 prior to addition of the alumina forming monomer and, optionally, PEG (e.g., PEG with molecular weight between 0.1 k and 1 k and concentration between 10 mM and 75 mM) is added to the reaction mixture prior to adjusting the pH to a pH of 7 to 9, and the nanoparticles are aluminosilicate nanoparticles.
Statement 29. A method according to Statement 27 or 28, where 1 to 7 (an average of 1 to 7, e.g., 2) photosensitizer molecules (e.g., one or more photosensitizer groups) are present in each of the nanoparticles (e.g., silica nanoparticle or aluminosilicate particles) surface functionalized with PEG groups.
Statement 30. A method according to any one of Statements 27-29, where the PEG-silane conjugate comprises a ligand described herein (e.g., a peptide (natural or synthetic), such as, for example, cyclic-RGD and derivatives thereof, alpha-MSH and derivatives thereof, and the like, a ligand comprising a moiety comprising a radio label (e.g., ¹²⁴I, ¹³¹I, ²²⁵Ac or ¹⁷⁷Lu, ⁸⁹Zr, ⁶⁴Cu, and the like), antibody, antibody fragment, targeting glycan (e.g., sugar molecules targeting cell surface receptors), nucleic acid (e.g., single stranded or double stranded DNA, various forms of RNA (e.g., siRNA, and the like), lipid, or carbohydrate (e.g., oligosaccharide, polysaccharide, sugar, and the like), drug, or combinations thereof), a ligand comprising a reactive group (e.g., a reactive group that can be conjugated to a molecule such a drug molecule) conjugated to a terminus of the PEG moiety opposite the terminus conjugated to the silane moiety (can be formed using a heterobifunctional PEG compound), or a combination thereof).
Statement 31. A method according to Statement 30, where the PEG-silane conjugate comprising a ligand is added in addition to PEG-silane in d), whereby nanoparticles surface functionalized with PEG groups and polyethylene groups comprising a ligand are formed.
Statement 32. A method according to any one of Statements 27-31, where before or after the PEG-silane conjugate is added in d) a PEG-silane conjugate comprising a ligand (e.g., at concentration between 0.05 mM and 2.5 mM) (e.g., PEG-silane conjugate comprising a ligand dissolved in a polar aprotic solvent such as, for example, DMSO or DMF) is added at room temperature to the reaction mixture comprising the core nanoparticles from b), holding the resulting reaction mixture at a time (t⁴) and temperature (T⁴) (e.g., (t⁴) 0.5 minutes to 24 hours at room temperature (T⁴)) (whereby at least a portion of the PEG-silane conjugate molecules are adsorbed on at least a portion of the surface of the nanoparticles from b)), subsequently heating the resulting reaction mixture at a time (t⁵) and temperature (T⁵) (e.g., (t⁵) 1 hour to 24 hours at 40° C. to 100° C. (T⁵)), whereby nanoparticles surface functionalized with PEG groups comprising a ligand are formed, optionally, subsequently adding at room temperature to the resulting reaction mixture comprising nanoparticles surface functionalized with PEG groups comprising a ligand a PEG-silane conjugate (the concentration of PEG-silane no ligand is between 10 mM and 75 mM) (e.g., PEG-silane conjugate dissolved in a polar aprotic solvent such as, for example, DMSO or DMF), holding the resulting reaction mixture at a time (t⁶) and temperature (T⁶) (e.g., (t⁶) 0.5 minutes to 24 hours at room temperature (T⁶)) (whereby at least a portion of the PEG-silane conjugate molecules are adsorbed on at least a portion of the surface of the nanoparticles surface functionalized with PEG groups comprising a ligand), and heating the resulting mixture from at a time (t⁷) and temperature (T⁷) (e.g., (t⁷) 1 hour to 24 hours at 40° C. to 100° C. (V)), whereby nanoparticles surface functionalized with PEG groups and PEG groups comprising a ligand are formed.
Statement 33. A method according to any one of Statements 27-32, where at least a portion of or all of the PEG-silane has a reactive group on a terminus of the PEG moiety opposite the terminus conjugated to the silane moiety of the PEG-silane conjugate (is formed from a heterobifunctional PEG compound) and after formation of the nanoparticles surface functionalized with PEG groups having a reactive group, and, optionally, PEG groups, are reacted with a second ligand (which can be the same or different than the ligand of the nanoparticles surface functionalized with PEG groups and PEG group comprising a ligand) functionalized with a second reactive group (which can be the same or different than the reactive group of the nanoparticles surface functionalized with PEG groups and PEG group comprising a ligand) thereby forming nanoparticles surface functionalized with polyethylene groups functionalized with a second ligand and, optionally, PEG groups.
Statement 34. A method according to Statement 32 or 33, where at least a portion of or all of the PEG-silane has a reactive group on a terminus of the PEG moiety opposite the terminus conjugated to the silane moiety of the PEG-silane conjugate (is formed from a heterobifunctional PEG compound) and after formation of the nanoparticles surface functionalized with PEG groups and, optionally, having a reactive group, and, optionally, PEG groups, are reacted with a second ligand (which can be the same or different than the ligand of the nanoparticles surface functionalized with PEG groups and PEG group comprising a ligand) functionalized with a second reactive group (which can be the same or different than the reactive group of the nanoparticles surface functionalized with PEG groups and PEG group comprising a ligand) thereby forming nanoparticles surface functionalized with polyethylene groups functionalized with a second ligand and, optionally, PEG groups, where at least a portion of the PEG-silane has a reactive group on a terminus of the PEG moiety opposite the terminus conjugated to the silane moiety of the PEG-silane conjugate (is formed from a heterobifunctional PEG compound) and after formation of the nanoparticles surface functionalized with PEG groups having a reactive group, nanoparticles surface functionalized with PEG groups having a reactive group and PEG groups comprising a ligand) thereby forming nanoparticles surface functionalized with PEG groups and polyethylene groups functionalized with a second ligand, nanoparticles surface functionalized with PEG groups comprising a ligand.
Statement 35. The method of any one of Statements 27-34, where the method further comprises one or more post-synthesis processes.

The following example is presented to illustrate the present disclosure. It is not intended to limiting in any matter.

### Example 1

This example provides a description of nanoparticles of the present disclosure.

MATERIALS. Aluminum-tri-sec-butoxide (ASB), (3-aminopropyl)triethoxysilane (APTES), ammonium hydroxide (28 wt % in H₂O), ammonia solution (2.0 M in ethanol), dimethyl sulfoxide (DMSO), 1,3-diphenylisobenzofuran (DPBF, 97%), hydrochloric acid (HCl, 0.5018 N in H₂O), (3-iodopropyl)trimethoxysilane (IPTMS), methylene blue (MB), (3-mercaptopropyl) trimethoxysilane (MPTMS), 2-propanol (anhydrous 99.5%), and tetramethyl orthosilicate (TMOS) were purchased from Sigma Aldrich. (3-aminopropyl)trimethoxysilane (APTMS) and methoxy-terminated poly(ethylene glycol) (PEG-silane, molar mass of ˜0.5 kg/mol) were purchased from Gelest. Heterobifunctional PEG (NHS-PEG-mal, molar mass of ˜960 g/mol) was purchased from Quanta BioDesign. ATTO MB2-maleimide was purchased from ATTO-Tec. Tetramethylrhodamine-5-maleimide (TMR) was purchased from AnaSpec. Ethanol (absolute anhydrous 99.5%) was purchased from Pharmco-Aaper. c(RGDyC) was purchased from Peptide International. Deionized (DI) water (18.2 MΩ·cm) was generated using a Millipore Milli-Q system. All chemicals were used as received.

SYNTHESIS OF NANOPHOTOSENSITIZERS (DESIGN ONE). First, 3.67×10⁻⁷ moles MB2 with a maleimide group are reacted with MPTMS in DMSO at a molar ratio of 1:25 (photosensitizer:MPTMS) to generate a MB2-silane conjugate. To synthesize sub-10 nm PEGylated SNPs with MB2 inside the silica core, 2 mL of 0.02 M ammonia aqueous solution is first added into 8 mL of DI water yielding a pH of ˜9. The solution is then stirred at room temperature for 5 min. As the silica precursor, 0.43 mmol of TMOS are added under vigorous stirring, followed by the addition of all MB2-silane. The molar ratio of MB2-silane to TMOS is about 1:1000. The solution is left stirring at room temperature overnight. Then, 0.21 mmol of PEG-silane are added and the solution is kept stirring at room temperature overnight. Finally, to promote covalent bond formation between PEG-silane and particles, stirring is stopped and the particle dispersion is heated to 80° C. for 8 hours. To remove any unreacted precursors, aggregates, or dust from the particle dispersion, particles are transferred into a dialysis membrane tube (molecular weight cutoff, MWCO=10,000), and dialyzed in 2 L of DI water with three water exchanges every 8 hours. After dialysis, the dispersion is subject to syringe filtration (0.2 μm, Fisherbrand) and finally up-concentrated for gel permeation chromatography (GPC) using a membrane spin filter (GE Healthcare, molecular weight cutoff=30,000) and a centrifuge at 2300 rpm.

SYNTHESIS OF NANOPHOTOSENSITIZERS (DESIGN TWO). Particles binding MB2 to the particle surface are synthesized according to the synthesis of design one (excluding the addition of MB2-silane, or replacing MB2 with TMR-maleimide, see main text). MB2 is added to the final silica particles by using the method of post-PEGylation surface modifications by insertion (PPSMI). To that end, MPTMS is added to the PEGylated particle dispersion under vigorous stirring at a concentration of 2.3 mM. The particle/MPTMS mixture is stirred at room temperature overnight, followed by the addition of 3.67×10⁻⁷ moles MB2-maleimide at a concentration of 37 μM. The solution is vigorously stirred at room temperature for 24 hours for the dye to react with the thiol group on the silica core surface of the particles. Afterwards, the particle dispersion is subjected to the same cleaning process as described before (dialysis, syringe filtration, GPC). Particles containing TMR on the surface were synthesized in the same way by replacing MB2-maleimide with TMR-maleimide.

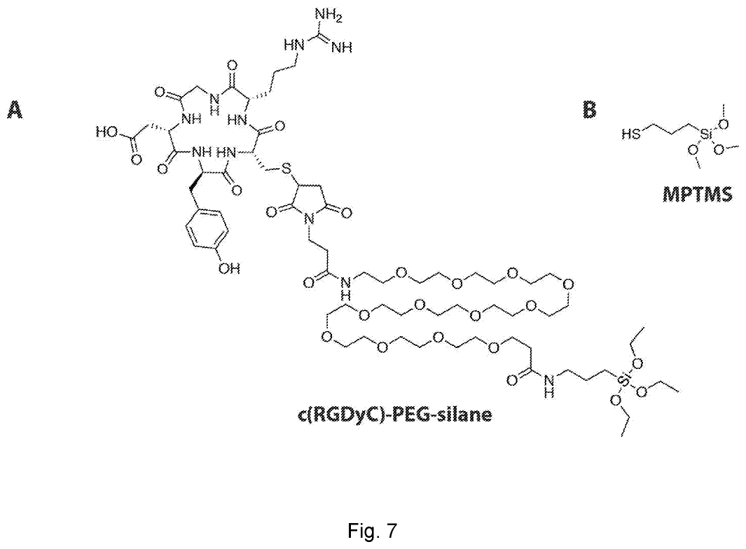

TARGETING PEPTIDE C(RGDYC) FUNCTIONALIZATION. Particles were peptide-functionalized with c(RGDyC)-PEG-silane (FIG. 7). c(RGDyC)-PEG-silane was prepared by exploiting the mercapto group of cysteine of c(RGDyC) (FIG. 1D) to click to the maleimide group of a heterobifunctional mal-PEG-NHS first, and then clicking the NHS to the amine group of (3-aminopropyl)trimethoxysilane (APTES). The concentration of NHS ester-PEG-maleimide in DMSO was 0.23 M. The mixed solution was left at room temperature in the glovebox for 3 hours to form silane-PEG-maleimide. After that, c(RGDyC) was added and the solution left at room temperature in the glovebox overnight to produce c(RGDyC)-PEG-silane. The molar ratio c(RGDyC):NHS-PEG-mal:APTES was 1.0:1.0:0.9. In order to functionalize particles with c(RGDyC) peptide ligands, previously prepared c(RGDyC)-PEG-silane was added to the particle dispersion immediately before the addition of PEG-silane. The remainder of the synthesis and purification protocol is as described before.

GEL PERMEATION CHROMATOGRAPHY (GPC). To remove unreacted precursors from the native particle dispersion, samples were purified using gel permeation chromatography (GPC). A BioLogic LP system with 275 nm UV detector and cross-linked copolymer of allyl dextran and N,N′-methylene bisacrylamide (Sephacryl S-300 HR, GE Healthcare) as solid phase was used. Before GPC purification each sample was up-concentrated with centrifuge spin-filters (Vivaspin with MWCO 30 k, GE Healthcare) to an approximate sample volume of 600 μL, run through the column with a 0.9 wt % NaCl solution, and fraction-collected by a BioFrac fraction collector. Sample fractions were transferred to DI water by washing samples five times with centrifuge spin-filters. The resulting particles could be subjected to long-term storage in nitrogen bubbled DI water in the dark at 4° C.

STEADY STATE ABSORPTION SPECTROSCOPY. Absorbance spectra were measured on a Varian Cary 5000 spectrophotometer. Spectra were measured in DI water using a quartz cuvette (HellmaAnalytics) with a 10 mm light path, and baseline corrected using a second cuvette with pure DI water as a reference cell. All spectra were measured in 1 nm increments and peak intensities were kept between 0.01 and 0.06.

FLUORESCENCE CORRELATION SPECTROSCOPY (FCS). A confocal FCS setup was used to determine particle hydrodynamic diameter, solution concentration, and number of dye molecules per particle. Particles containing TMR dye were excited with a 543 nm He:Ne laser, that was focused by a water immersion microscope objective (Zeiss Plan-Neofluar 63× NA 1.2). The fluorescence signal passed through a 50 μm pinhole and a long pass filter (ET5601p, Chroma) before being detected by an avalanche photo diode (APD) detector (SPCM-AQR-14, PerkinElmer) and auto-correlated with a digital correlator (Flex03LQ, Correlator.com). Data was fitted using a non-linear least-squares Levenberg-Marquardt algorithm and a triplet corrected correlation function, G(τ), shown in equation (1):

$$G ( τ ) = 1 + \frac{1}{{N}_{m}} ( \frac{1}{1 + τ \text{/} {τ}_{D}} ) {( \frac{1}{1 + τ \text{/} ( {τ}_{D} {κ}^{2} )} )}^{1 \text{/} 2} \frac{1}{( 1 - T )} ( 1 - T + T exp ( τ \text{/} {τ}_{T} ) ) \qquad ( 1 )$$

Where τ is the lag time, Nₘ the time- and spaced-averaged number of TMR labeled particles in the FCS observation volume, that is defined by a structure factor κ=ω_{z}/ω_{xy} with radial (ω_{xy}) and axial (ω_{z}) radii. τ_{D} is the time that a particle takes to diffuse through the observation volume. T is the fraction of TMR molecules being in the triplet state, with a triplet relaxation time, τ_{T}. FCS correlation curves were normalized using equation (2):

G(τ)=(G(τ)−1)Nₘ (2)

All samples were measured in 35 mm glass bottom dishes (P35G-1.5-10-C, Mattek Corporation) at nanomolar concentration in DI water at 20° C., 5 kW cm⁻² laser power, and in triplets with five 30 s long collection intervals. The observation volume was calibrated before each FCS measurement. Particle diameters, d, were calculated using the Stokes-Einstein equation (3) with the diffusion constant, D, obtained from equation (4):

$$\begin{matrix}d = 2 \frac{{k}_{B} T}{6 πη D} & ( 3 ) \\ D = \frac{{ω}_{xy}^{2}}{4 {τ}_{D}} & ( 4 )\end{matrix}$$

The number of TMR or MB2 molecules per particle, nₘ, was determined by comparing the dye concentration from steady state absorption spectroscopy, C_{Abs}, and the particle concentration measured in FCS, <C>_{FCS}, using equation (5):

$${n}_{m} = \frac{{C}_{Abs}}{{〈 C 〉}_{FCS}} \qquad ( 5 )$$

where it was assumed that the molar extinction coefficients do not change upon dye encapsulation.

DETERMINATION OF SINGLET OXYGEN QUANTUM YIELDS, Φ_{Δ}. Singlet oxygen quantum yield, Φ_{Δ}, measurements were carried out in ethanol with 1,3-diphenylisobenzofuran (DPBF) as a detector molecule for trapping singlet oxygen. The generation of singlet oxygen could be observed by a reduction of the DPBF absorption band at 410 nm (FIG. 3A). Measurements were carried out at sample optical densities of 0.15-0.50 in a 100 μL quartz cuvette (Starna). Samples were evenly exposed to a 635 nm, expanded, and collimated laser beam of a solid-state laser (Power Technology Inc.) at 3 mW/cm² with a spot size of about 1 cm in the same cell. To acquire a 0.5-0.6 absorption, DPBF was added at a concentration of approximately 18.75 μM. All absorption spectra were measured in 1 nm steps and baseline-corrected against a second cuvette with ethanol as a reference cell. The sample absorption was recorded at defined time intervals and corrected for the sample absorption spectrum in the absence of DPBF. Φ_{Δ} was calculated by comparing all samples to the standard methylene blue (MB) dye with known singlet oxygen quantum yield of Φ_{Δ}=0.52 (in ethanol) (31) by plotting the natural logarithm of the reduction of the 410 nm DBPF band against the exposure time and using equation (6), where m represents the slope of a linear fit through the data points (FIG. 3B):

$${Φ}_{Δ} ( sample ) = {Φ}_{Δ} ( MB ) \frac{m ( sample )}{m ( MB )} \qquad ( 6 )$$

To determine the effective singlet oxygen quantum yield, Φ_{Δ}^{eff}, the particle concentration as determined by FCS and the MB concentration were matched.

Silica nanophotosensizers (SNPSs) covalently encapsulating the methylene blue derivate MB2 inside the particle (design one, FIG. 1C) were synthesized by combining tetramethylorthosilcate (TMOS) and MB2-silane (FIG. 1B) in basic aqueous solution. After particle formation, further particle growth was quenched by the addition of PEG-silane (FIG. 1B) to the reaction mixture. Particles containing MB2 on the particle surface (design two, FIG. 1C) were synthesized in the same way, however, MB2 was attached using a grafting method referred to as post-PEGylation surface modification by insertion (PPSMI). This method employs amine-reactive or sulfhydryl-reactive click chemistry, by adding amine-silanes or thiol-silanes, respectively, below the nucleation threshold into an aqueous dispersion of PEGylated SNPs. The small molar mass silane precursors diffuse through the PEG corona chains and react with the silica particle surface. The pending amine or thiol groups can further be reacted with N-hydroxysuccinimide or maleimide functional groups, respectively. For design two, we used (3-mercaptopropyl)trimethoxysilane (MPTMS) to functionalize the particle surface with thiol groups to click MB2-maleimide to the particle (for details see Materials and Methods). Finally, all particles were cleaned from unreacted precursors via gel permeation chromatography (GPC) prior to further characterization.

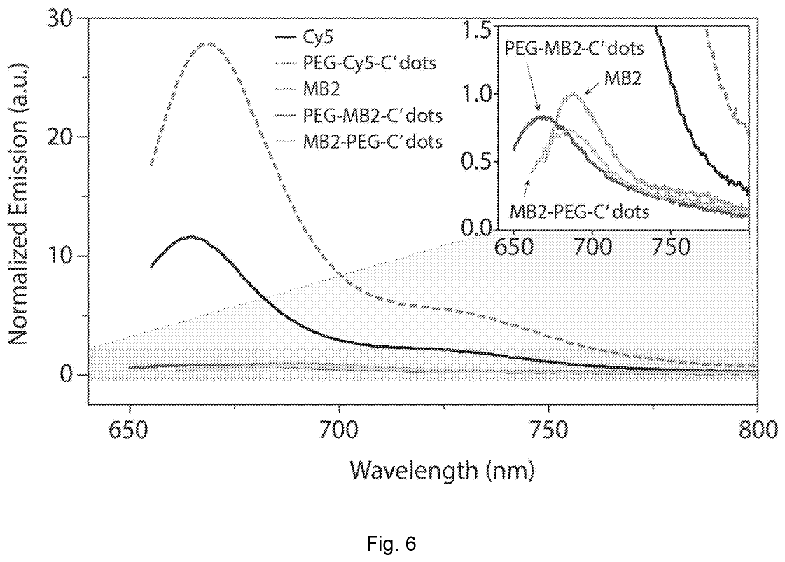

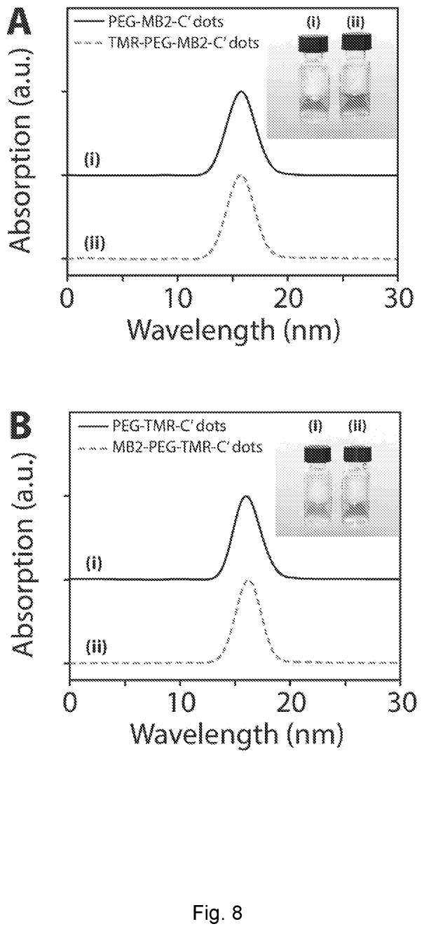

Due to the weakly-emissive nature of MB2 (FIG. 6), fluorescent-based size determination of MB2 functionalized particles by fluorescence correlation spectroscopy (FCS) was not possible. To make particle samples accessible for the determination of hydrodynamic diameters and particle concentrations by FCS, particles were further functionalized with the fluorescent dye tetramethylrhodamine-silane (TMR-silane) (FIG. 1B). For design one we grafted TMR onto the particle surface using PPSMI and for design two we synthesized SNPs encapsulating TMR dye before MB2 was grafted on the particle surface. A combination of FCS and steady state absorption spectroscopy was used to determine the number of MB2 and TMR molecules per particle. Particle diameter and concentration were determined by measuring the fluorescence fluctuations of particles diffusing through a well-defined observation volume of a laser beam and subsequently auto-correlating the fluorescence time signal. The resulting FCS correlation curves were fitted with a correlation function (see equation (1), Materials and Methods) from which the time averaged number of particles and the diffusion constant were extracted. To determine the number of dyes per particle, the dye concentration as determined by steady-state absorption spectroscopy was compared to the concentration of the particles as determined by FCS (equation (5), Materials and Methods), yielding the average number of dyes per particle. Dye molecules that were not covalently bound during synthesis were washed away by dialysis and separated from the particles by GPC. FIGS. 8A and 8B show the GPC elugrams before and after TMR and MB2 surface functionalization of particles, respectively. Both elugram-pairs show a single peak and were congruent to each other, indicating that TMR dye molecules (design one), or MB2 dye molecules (design two) were grafted onto the respective SNPs.

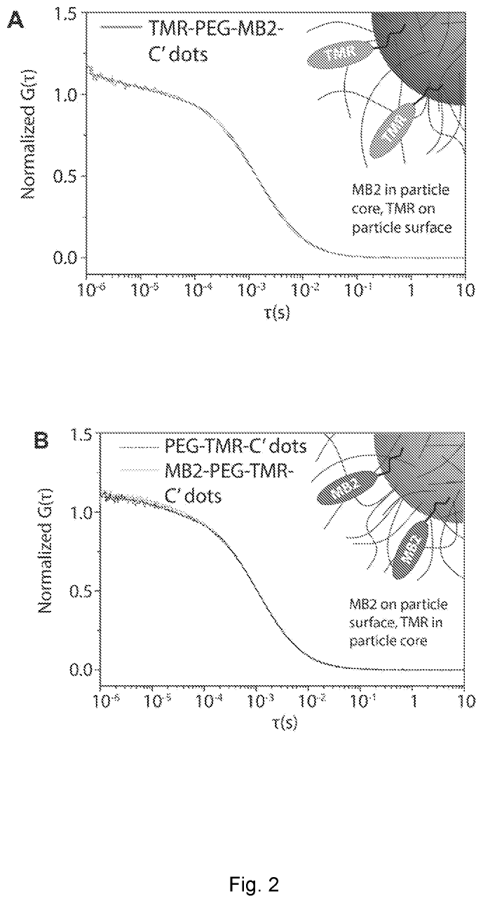

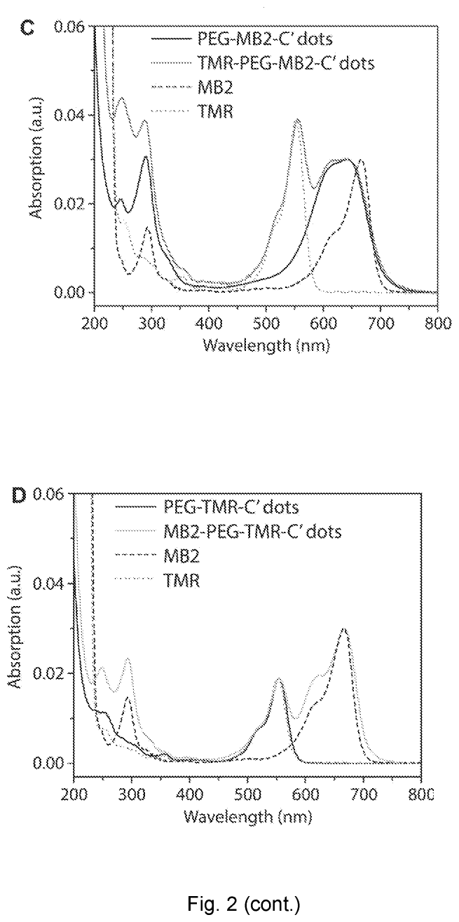

FIGS. 2A and 2B show the FCS correlation curves of the fluorescent particles containing the photosensitizer MB2. The curves were fitted using a triplet corrected translational diffusion correlation function (equation (1), Materials and Methods). Particle hydrodynamic diameters of 5.9 nm for MB2 encapsulating C′ dots with TMR surface functionalization (TMR-PEG-MB2-C′ dots), 5.2 nm for TMR encapsulating C′ dots (PEG-TMR-C′ dots), and 5.2 nm for TMR encapsulating C′ dots with MB2 surface functionalization (MB2-PEG-TMR-C′ dots) were obtained. FIGS. 2C and 2D show the UV-vis absorption spectra of PEG-MB2-C′ dots, TMR-PEG-MB2-C′ dots, PEG-TMR-C′ dots, and MB2-PEG-TMR-C′ dots in water, respectively. For comparison, the absorption spectra of TMR-maleimide and MB2-maleimide are superimposed onto the particle spectra. For TMR-PEG-MB2-C′ dots and MB2-PEG-TMR-C′ dots, a TMR absorption peak can be observed indicating successful functionalization of particles with TMR and MB2, respectively.

Comparing the absorption profiles of MB2 for the two different designs, a relative hypsochromic shift (blue-shift) from 668 nm to 644 nm of the main peak for design one relative to free MB2 is observed that is absent in design two. This hypsochromic shift likely originates from dimethylation of the auxochrome groups of MB2, from —N(CH₃)₂ to —NH(CH₃) and/or —NH₂, which is promoted in basic media. In addition, both designs display a heightened left shoulder in the absorption peak as compared to free MB2 dye that is more pronounced in design one than it is in design two. The heightened shoulders around 620 nm and 605 nm for design two and one, respectively, are a result of dimerization of MB2 at high concentrations (1×10⁻⁶ to 4×10⁻⁴ M) in aqueous media (MB2 concentrations during synthesis is 3.67×10⁻⁵ M) (34). MB monomers and dimers are known to have distinct absorption peaks located at 664 nm and 590 nm, respectively, with an equilibrium constant of 3.8×10³ M⁻¹ in water. However, the formation of dimers is not only dependent on concentration but is additionally promoted by the presence of oppositely charged surfaces. For design one, the cationic MB2 sensitizer was added to the synthesis during the silica particles formation and hence was exposed to negatively charged silica nucleation seeds (at pH 9). For design two, MB2 was grafted onto the PEGylated silica particle surface at neutral conditions (pH 7), consequently showing no peak shift and relatively fewer MB2 dimers, despite the same MB2 concentration during the synthesis as for design one.

To determine the number of MB2 molecules per particle we compared the particle concentrations estimated by FCS and the MB2 concentrations from steady-state absorption measurements. For practical reasons, we assumed that the extinction coefficient remained unaffected in the particle synthesis. This is not necessarily true due to the metachromatic nature of methylene blue and demethylation. Based on this assumption we estimated the average number of dyes per particle (equation (5), Materials and Methods) to be 2.4/3.3 for MB2/TMR for design one and 3.4/2.3 for MB2/TMR for design two.

Next, we measured the singlet oxygen quantum yield, Φ_{Δ}, for both particle designs using the singlet oxygen sensor 1,3-diphenylisobenzofuran (DPBF). For these measurements, we matched the particle concentrations as determined by FCS to yield an effective singlet oxygen quantum yield per SNPS (Φ_{Δ}^{eff} (SNPS)). FIG. 3A demonstrates the principle of oxygen sensing using DPBF and the particle TMR-PEG-MB2-C′ dots (design one) in ethanol. The mixture is evenly exposed to an expanded and collimated 635 nm laser beam for defined time intervals. The singlet oxygen that is generated by the SNPSs reacts with DPBF molecules, yielding 1,2-dibenzoylbenzene. The formation of 1,2-dibenzoylbenzene was monitored via a reduction of the absorption band at 410 nm. By comparing samples to a methylene blue standard (Φ_{Δ}(MB)=0.52), Φ_{Δ}^{eff}(SNPS) was determined (see equation (6), Materials and Methods), resulting in values of 111% for design one (TMR-PEG-MB2-C′ dots) and 161% for design two (MB2-PEG-TMR-C′ dots). This translates to an estimated per dye singlet oxygen quantum yield of 46% and 47%, respectively, based on the estimated number of MB2 dyes per particle. The relatively lower values for Φ_{Δ} of the dyes associated with the particles versus free methylene blue dye can be rationalized by the steric shielding effects of encapsulation or grafting within the PEGylation corona. The silica network and/or the PEG molecules shield diffusing oxygen, first, from MB2, and then, from DPBF resulting in a reduced singlet oxygen quantum yield. Although for both designs the per dye Φ_{Δ} values are similar, surface grafted MB2 molecules are likely less shielded than dyes fully encapsulated in the silica network. In addition, it is known that methylene blue dimers and monomers engage in different photochemical processes. While monomers undergo energy transfer reaction with triplet oxygen, dimers engage in electron transfer reactions with other methylene blue molecules. These different energy dissipation pathways of dimers correlate negatively with the singlet oxygen quantum yield contributing to the reduced singlet oxygen quantum yield per dye (e.g., PS) molecule of design one and design two. However, the multiplicity effect stemming from multiple MB2 molecules colocalized on one particle compensates for a reduced per dye (e.g., PS) singlet oxygen quantum yield by steric shielding and/or dimerization.

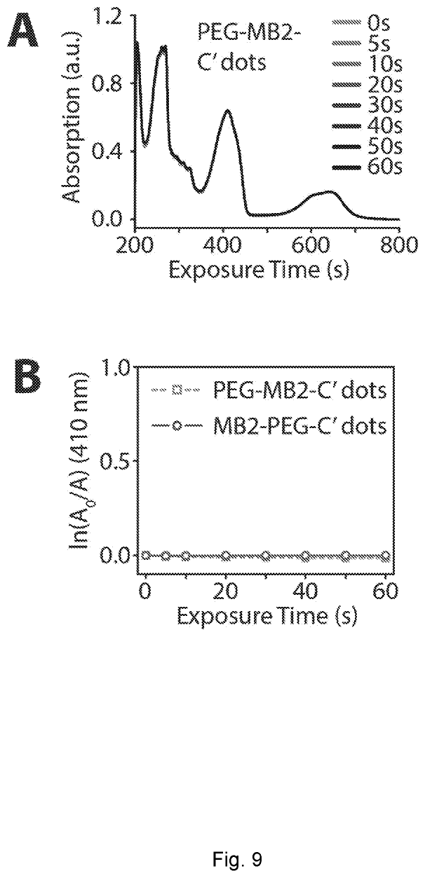

To exclude the possibility of ¹O₂ formation in the absence of irradiation with light (dark toxicity) for the different particle designs, we repeated singlet oxygen quantum yield measurements, but did not expose the samples to the laser beam. FIG. 9 shows results of the same experiment as shown in FIG. 3A for design one (PEG-MB2-C′ dots). This time, the DPBF peak at 410 nm remains unchanged, however, indicating no formation of 1,2-dibenzoylbenzene and hence no generation of singlet oxygen. This is the case for both designs.

Specific targeting of photosensitizers to diseased tissue increases the efficacy of PDT and minimizes collateral damage to healthy tissue. We therefore functionalized SNPSs with the targeting moiety cyclo(arginine-glycine-aspartic acid-D-tyrosine-cysteine) (c(RGDyC)) (FIG. 1C), which targets αᵥβ₃ integrins overexpressed, e.g., on various cancer cells including melanoma. It has been shown that the endocytosis-mediated cellular uptake of c(RGDyC) functionalized particles correlates with the αᵥβ₃-expression levels of cells, and increases the intracellular particle concentration, rendering c(RGDyC) a specific targeting moiety with high affinity for the treatment of the melanoma cancer.

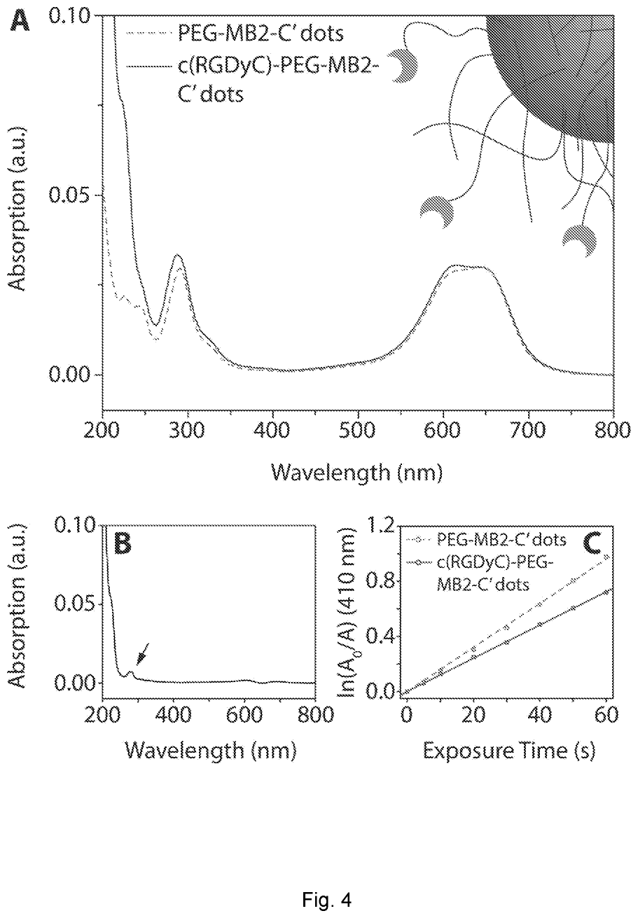

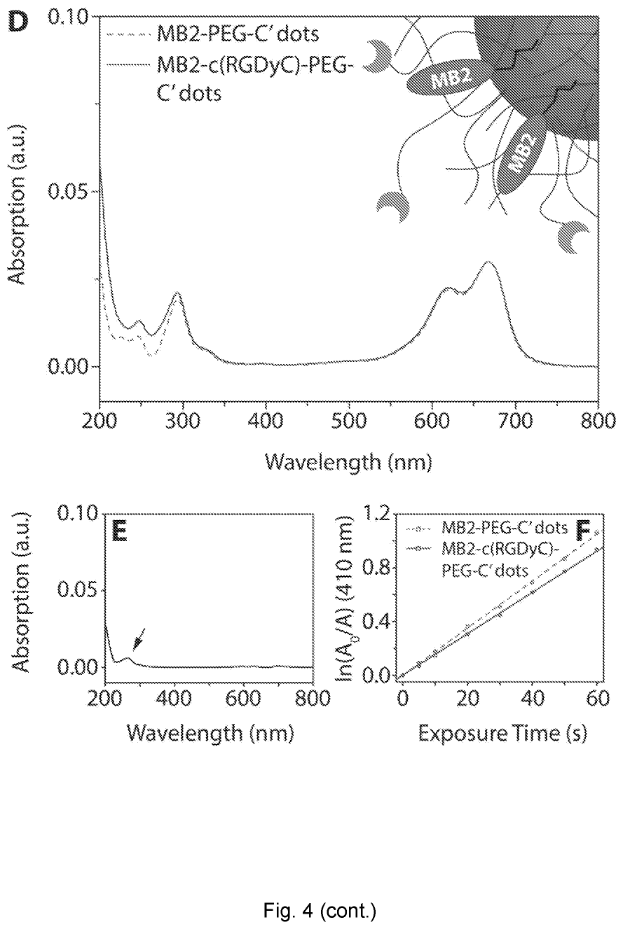

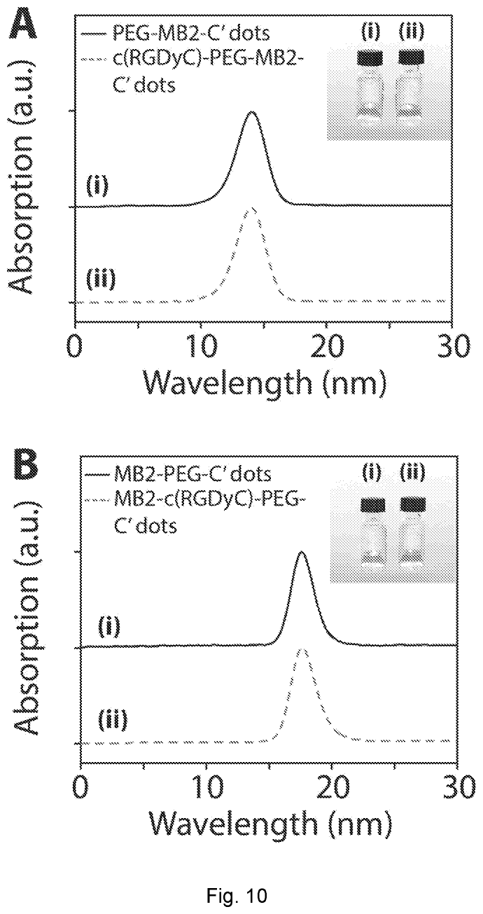

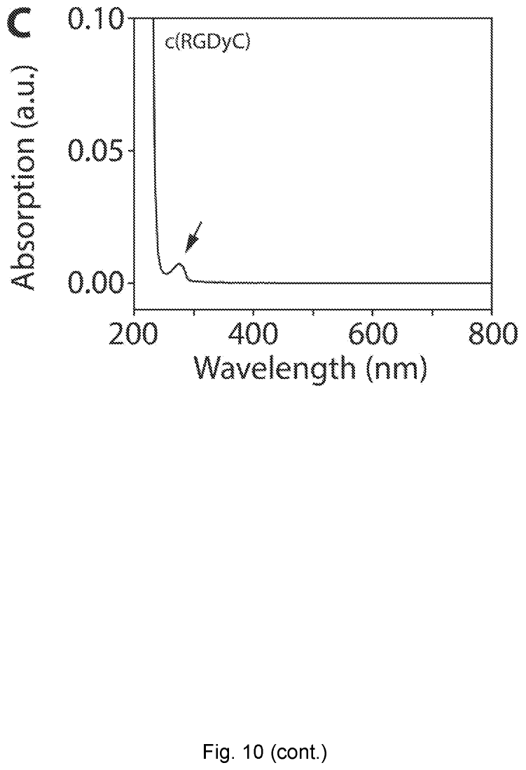

Particles were functionalized by adding c(RGDyC)-PEG-silane (FIG. 7A) during the PEGylation step. To allow more steric freedom for ligand binding to integrins, the c(RGDyC)-PEG-silane was chosen to be three ethylene oxide (EO) units longer than the PEG-silane (twelve versus six-nine units). Due to the weakly-fluorescent nature of MB2, a FCS analysis could not be conducted. Instead we compared the GPC elugrams before and after peptide functionalization for particle design one (PEG-MB2-C′ dots and c(RGDyC)-PEG-MB2-C′ dots), and two (MB2-PEG-C′ dots and MB2-c(RGDyC)-PEG-C′ dots) (FIGS. 10A and 10B). In both cases, we observed single peaks that were congruent to each other. All particles were then characterized using steady-state absorption spectroscopy. FIGS. 4A and 4D show the absorption spectra of design one and design two, respectively, with and without c(RGDyC)-functionalization in water. In both cases, increased absorption between 200 and 300 nm was noticeable. Due to strong absorption features in that region it is difficult to clearly identify the peptide absorption by qualitative comparison. For that reason, we subtract the two spectra from each other and display the difference spectra in FIGS. 4B and 4E. In both cases a band at ˜260 to 270 nm can clearly be identified, which coincides with the absorption band of the c(RGDyC) spectrum (FIG. 10C). Using the relative absorption peaks of tyrosine in c(RGDyC) and of MB2, we estimated 17 and 14 c(RGDyC) units per MB2 molecule for design one and design two, respectively, which is close to the desired number based on earlier studies.

Finally, we tested the effect of c(RGDyC)-functionalization on the relative singlet oxygen quantum yield performance. We compared particles with and without c(RGDyC) for absorption matched samples of the same design. For both designs we measured a reduction of singlet oxygen quantum yield by a relative 25% for design one and by a relative 12% for design two (FIGS. 4C and 4F). This finding is surprising. Given the spatial proximity of surface MB2 and c(RGDyC), one would expect a stronger effect of c(RGDyC) in design two. Results suggest, however, that c(RGDyC) increases the steric shielding more significantly for the encapsulated MB2 than for the surface grafted MB2.

Although the present disclosure has been described with respect to one or more particular embodiments and/or examples, it will be understood that other embodiments and/or examples of the present disclosure may be made without departing from the scope of the present disclosure.

1. A composition comprising a plurality of silica nanoparticles and/or aluminosilicate nanoparticles,
wherein the individual nanoparticles comprise silica cores or aluminosilicate cores and the silica cores or aluminosilicate cores are surface functionalized with polyethylene glycol (PEG) groups and comprise at least one photosensitizer group, and
at least 90%, 95%, 96%, 97%, 98%, 99%, 99.5%, or 99.9% or 100% of the nanoparticles have a size of 2 to 9.99 nm.

2. The composition of claim 1, wherein all of the at least one photosensitizer groups are the same.

3. The composition of claim 1, wherein the individual nanoparticles comprise two or more photosensitizer groups and the at least two of the photosensitizers groups are structurally different photosensitizer groups.

4. The composition of claim 1, wherein the individual nanoparticle cores comprise 1 to 7 photosensitizer groups.

5. The composition of claim 1, wherein the individual nanoparticle cores comprise 1 to 7 photosensitizer groups and the nanoparticles have 1 to 30 photosensitizers covalently bound to a surface of the nanoparticle core and/or photosensitizers that are part of a PEG group.

6. The composition of claim 1, wherein the at least one photosensitizer group is chosen from psoralen groups, porphyrinoid groups, phenothiazine groups, cyanine groups, curcuminoid groups, boron-dipyrromethene (BODIPY) groups, xanthene groups, derivatives thereof, and combinations thereof.

7. The composition of claim 1, wherein the photosensitizer is covalently attached to the silica or aluminosilicate core matrix via a thioether linkage.

8. The composition of claim 1, wherein the individual nanoparticles have at least one photosensitizer group completely or partially encapsulated within the nanoparticle.

9. The composition of claim 1, wherein the at least one photosensitizer group is disposed on the surface of the nanoparticle or is part of a PEG group.

10. The composition of claim 1, wherein the photosensitizer group(s) of the individual nanoparticles is/are completely encapsulated within the individual nanoparticles, partially encapsulated within the individual nanoparticles, disposed on the surface of the individual nanoparticles, are part of a PEG group of the individual nanoparticles, or a combination thereof.

11. The composition of claim 1, wherein all of the photosensitizer group(s) of the individual nanoparticles is/are completely encapsulated within the individual nanoparticles.

12. The composition of claim 11, wherein the nanoparticles do not exhibit detectible surface presence of the photosensitizer(s) as determined by high-performance liquid chromatography (HPLC).

13. The composition of claim 1, wherein at least a portion or all of the nanoparticles further comprise one or more functional group chosen from fluorescent dyes, chelators for radio-isotopes, targeting groups, drugs, and combinations thereof, wherein the one or more functional group is covalently bound to a surface of the nanoparticle(s), part of a PEG group, or a combination thereof.

14. The composition of claim 1, wherein at least a portion or all of the PEG groups comprise one or more ligand group, wherein the ligand group is disposed on a surface of the nanoparticles and/or is part of a PEG group.

15. The composition of claim 14, further comprising one or more radioisotope attached to the ligand group(s).

16. The composition of claim 15, wherein the radioisotope(s) is/are therapeutic radioisotope(s).

17. The composition of claim 14, wherein the composition comprises one or more drug-linker conjugate covalently attached to the ligand group(s), wherein the linker group(s) is/are configured to be cleaved by an enzyme or acidic environment in a tumor.

18. The composition of claim 14, wherein the composition comprises one or more targeting group covalently attached to the ligand group(s).

19. The composition of claim 1, further comprising a pharmaceutically acceptable carrier.

20. The composition of claim 1, wherein the composition has not been subjected to any particle-size discriminating process or processes.

21. A method of treating an individual in need of treatment for cancer, comprising administering a composition of claim 1.

22. The method of claim 21, further comprising exposing the individual or a portion thereof to light having a wavelength of 400-900 nm.

23. The method of claim 21, wherein the nanoparticles comprise a drug and the drug is released in the individual.

24. The method of claim 23, wherein the drug is released in a selected portion of the individual.

25. The method of claim 21, further comprising imaging the individual.

26. The method of claim 25, wherein the imaging is fluorescence imaging.

27. A method of making nanoparticles of the surface functionalized with polyethylene glycol (PEG) groups, wherein at least 90%, 95%, 96%, 97%, 98%, 99%, 99.5%, or 99.9% or 100% of the nanoparticles have a size of 2 to 9.99 nm, comprising:
a) forming a reaction mixture at room temperature comprising:
water,
TMOS, and
a photosensitizer precursor,
wherein the pH of the reaction mixture is 6 to 9,
b) holding the reaction mixture for a selected time and temperature, whereby the nanoparticles are formed,
c) optionally, adjusting, the pH of the reaction mixture to a pH of 6 to 10 comprising the nanoparticles from b),
d) adding at room temperature to the reaction mixture comprising the nanoparticles from b) or c), a PEG-silane conjugate and holding the resulting reaction mixture for a selected time and temperature; and
e) optionally heating the mixture from d) at a selected time and temperature, whereby the nanoparticles surface functionalized with PEG groups are formed.

28. The method of claim 27, wherein the reaction mixture further comprises an alumina forming monomer and the pH of the reaction mixture is adjusted to a pH of 1 to 2 prior to addition of the alumina forming monomer and, optionally, PEG is added to the reaction mixture prior to adjusting the pH to a pH of 7 to 9, and the nanoparticles are aluminosilicate nanoparticles.

29. The method of claim 27, wherein 1 to 7 photosensitizer groups are present in each of the nanoparticles surface functionalized with PEG groups.

30. The method of claim 27, wherein the PEG-silane conjugate comprises a ligand conjugated to a terminus of the PEG group opposite the terminus conjugated to the silane group.

31. The method of claim 30, wherein the PEG-silane conjugate comprising a ligand is added in addition to PEG-silane in d), whereby nanoparticles surface functionalized with PEG groups and polyethylene groups comprising a ligand are formed.

32. The method of claim 27, wherein before or after the PEG-silane conjugate is added in d) a PEG-silane conjugate comprising a ligand is added at room temperature to the reaction mixture comprising the nanoparticles from b), holding the resulting reaction mixture at a selected time and temperature, subsequently heating the resulting reaction mixture at a selected time and temperature, whereby nanoparticles surface functionalized with PEG groups comprising a ligand are formed, optionally, subsequently adding at room temperature to the resulting reaction mixture comprising nanoparticles surface functionalized with PEG groups comprising a ligand a PEG-silane conjugate, holding the resulting reaction mixture at a selected time and temperature, and
heating the resulting mixture from at a selected time and temperature, whereby nanoparticles surface functionalized with PEG groups and PEG groups comprising a ligand are formed.

33. The method of claim 27, wherein at least a portion of or all of the PEG-silane has a reactive group on a terminus of the PEG group opposite the terminus conjugated to the silane group of the PEG-silane conjugate and after formation of the nanoparticles surface functionalized with PEG groups having a reactive group, and, optionally, PEG groups, are reacted with a second ligand functionalized with a second reactive group, thereby forming nanoparticles surface functionalized with polyethylene groups functionalized with a second ligand and, optionally, PEG groups.

34. The method of claim 32 or 33, wherein at least a portion of or all of the PEG-silane has a reactive group on a terminus of the PEG group opposite the terminus conjugated to the silane group of the PEG-silane conjugate and after formation of the nanoparticles surface functionalized with PEG groups and, optionally, having a reactive group, and, optionally, PEG groups, are reacted with a second ligand functionalized with a second reactive group, thereby forming nanoparticles surface functionalized with polyethylene groups functionalized with a second ligand and, optionally, PEG groups, or
wherein at least a portion of the PEG-silane has a reactive group on a terminus of the PEG group opposite the terminus conjugated to the silane group of the PEG-silane conjugate and after formation of the nanoparticles surface functionalized with PEG groups having a reactive group, nanoparticles surface functionalized with PEG groups having a reactive group and PEG groups comprising a ligand, thereby forming nanoparticles surface functionalized with PEG groups and polyethylene groups functionalized with a second ligand, nanoparticles surface functionalized with PEG groups comprising a ligand.

35. The method of claim 27, wherein the method further comprises one or more post-synthesis processes.
